(19) United States
(12) Patent Application Publication
(10) Pub. No.: US 2014/0371464 A1
(43) Pub. Date: Dec. 18, 2014

(54) PROCESS FOR THE PREPARATION OF CHIRAL ISOXAZOLINE AZETIDINE DERIVATIVES AS ANTIPARASITIC AGENTS

(71) Applicant: Zoetis LLC, Florham Park, NJ (US)

(72) Inventors: Denis Billen, Kalamazoo, MI (US); Sean David William Greenwood, Kalamazoo, MI (US); Timothy Lee Stuk, Kalamazoo, MI (US)

(21) Appl. No.: 14/375,056

(22) PCT Filed: Jan. 29, 2013
(86) PCT No.: PCT/US13/23673

§ 371 Date: Jul. 28, 2014

Related U.S. Application Data
(60) Provisional application No. 61/594,614, filed on Feb. 3, 2012.

(51) Int. Cl. C07D 413/10

(52) U.S. Cl. CPC C07D 413/10
USPC 548/240; 548/952; 548/953

(57) ABSTRACT

The invention recites a chiral process for the synthesis of isoxazoline azetidine phenyl substituted derivatives of Formula (1) stereoisomers thereof, veterinarily acceptable salts thereof, processes for making, and their use as a parasiticide in an animal. The variables *, R^{1a}, R^{1b}, R^{1c}, R², and R³ are as described herein

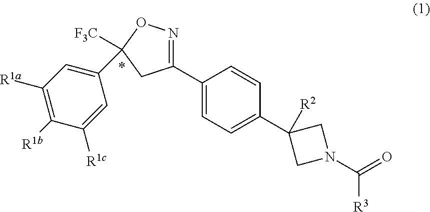

### FIELD OF THE INVENTION

This invention relates to the process of preparing isoxazoline azetidine derivatives as a pure enantiomer or enriched in a single enantiomer, compositions and methods of use thereof. The compounds of interest display parasiticidal activity.

### BACKGROUND

There is a need for improved antiparasitic agents for use with animals, and in particular there is a need for improved insecticides and acaricides. Furthermore there is a need for improved topical and oral products with convenient administration and which contain one or more of such antiparasitic agents which can be used to effectively treat ectoparasites, such as insects (e.g., fleas, lice, and flies) and acarids (e.g., mites and ticks). Such products would be particularly useful for the treatment of animals.

The compounds currently available for insecticidal and acaricidal treatment of animals do not always demonstrate good activity, good speed of action, or a long duration of action. Most treatments contain hazardous chemicals that can have serious consequences, including neurotoxicity and lethality from accidental ingestion. Persons applying these agents are generally advised to limit their exposure. Pet collars and tags have been utilized to overcome some problems, but these are susceptible to chewing, ingestion, and subsequent toxicological affects to the animal. Thus, current treatments achieve varying degrees of success which depend partly on toxicity, method of administration, and efficacy. Currently, some agents are actually becoming ineffective due to parasitic resistance.

Isoxazoline derivatives have been disclosed in the art as having insecticidal and acaricidal activity. For example, WO2007/105814, WO2008/122375, and WO2009/035004 recite certain alkylene linked amides. Further, WO2007/075459 discloses phenyl isoxazolines substituted with 5- to 6-membered heterocycles. Chiral processes for manufacturing isoxazolines have been reported in WO2011/104089 and WO2009/063910. However, none of these citations exemplify the processes of manufacturing phenylazetidine substituted isoxazolines, nor does the prior art indicate that such compounds would be useful against a spectrum of parasitic species relevant to companion animals, livestock or fowl against the range of parasitic morphological lifecycle stages. The fluoroazetidine isoxazolines were described in WO2012/017359, however, a chiral process was not described.

Despite the availability of effective, broad spectrum antiparasitic agents, there remains a need for a safer, convenient, efficacious, and environmentally friendly product that will overcome the ever-present threat of resistance development.

The present invention overcomes one or more of the various disadvantages of, or improves upon, the properties of existing compounds. In particular the present invention develops a process for the preparation of isoxazoline substituted azetidine compounds as a single enantiomer or significantly enriched in a single enantiomer, which demonstrate such properties.

### SUMMARY

The present invention provides a process for preparing the isoxazolines of Formula (1) as either a single enantiomer (i.e., pure) or significantly enriched enantiomer

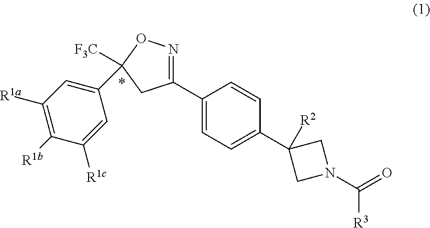

wherein

R^{1a}, R^{1b}, and R^{1c} are each independently hydrogen, halo, or C₁-C₆haloalkyl;

R² is hydroxyl, or fluoro;

R³ C₁-C₆alkyl, C₂-C₆alkenyl, C₀-C₆alkyIC₃-C₆cycloalkyl, C₀-C₆alkylphenyl, C₀-C₆alkylheteroaryl, or C₀-C₆alkylheterocycle;

wherein R³ C₁-C₆alkyl or C₀-C₆alkylC₃-C₆cycloalkyl moiety can be optionally and independently substituted by at least one substituent selected from cyano, halo, hydroxyl, oxo, C₁-C₆alkoxy, C₁-C₆haloalkoxy, C₁-C₆haloalkyl, —S(O)ₙR^{c}, —SH, —S(O)ₙNR^{a}R^{b}, —NR^{a}R^{b}, —NR^{a}C(O)R^{b}, —SC(O)R, —SCN, or —C(O)NR^{a}R^{b}, and the C₀-C₆alkylC₃-C₆cycloalkyl moiety can be further substituted with C₁-C₆alkyl or hydroxyl C₁-C₆alkyl-; and

wherein R³ C₀-C₆alkylphenyl, C₀-Colkylheteroaryl, or C₀-C₆alkylheterocycle moiety can be further optionally substituted with at least one substituent selected from cyano, halo, oxo, ═S, hydroxyl, C₁-C₆alkoxy, C₁-C₆alkyl, C₁-C₆haloalkyl, —SH, —S(O)ₙR, and C₁-C₆haloalkoxy;

R is C₁-C₆alkyl or C₃-C₆cycloalkyl optionally substituted with at least one halo substituent;

R^{a} is hydrogen, C₁-C₆alkyl, or C₀-C₃alkylC₃-C₆cycloalkyl; wherein the alkyl and alkylcycloalkyl is optionally substituted by cyano or at least one halo substituent;

R^{b} is hydrogen, C₁-C₆alkyl, C₃-C₆cycloalkyl, C₀-C₃alkylphenyl, C₀-C₃alkylheteroaryl, or C₀-C₃alkylheterocycle, each optionally substituted, where chemically possible, with at least one substituent selected from hydroxyl, cyano, halo, or —S(O)ₙR;

R^{c} is C₁-C₆alkyl, C₁-C₆haloalkyl, C₁-C₆haloalkylC₃-C₆cycloalkyl, C₀-C₃alkylC₃-C₆cycloalkyl, C₀-C₃alkylphenyl, C₀-C₃alkylheteroaryl, or C₀-C₃alkylheterocycle each optionally substituted with at least one substituent selected from cyano, halo, hydroxyl, oxo, C₁-C₆alkoxy, C₁-C₆haloalkoxy, C₁-C₆haloalkyl, —S(O)ₙR, —SH, —S(O)ₙNR^{a}R^{b}, —NR^{a}R^{b}, —NR^{a}C(O)R^{b}, —SC(O)R, —SCN, or —C(O)NR^{a}R^{b};

n is the integer 0, 1, or 2; and

* depicts a chiral center; stereoisomers thereof, and veterinarily acceptable salts thereof; said process comprising, optionally in a solvent:

a) metallating 1-bromo-4-iodobenzene with a Grignard reagent or halogen-metal exchange with an alkyllithium and reacting metallated species with a protected azetidinone in a one-pot process or in a step-wise process to provide a protected 3-(4-bromophenyl)azetidin-3-ol, wherein PG is an amine protecting group;

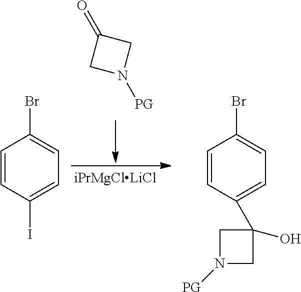

b) optionally fluorinating the resulting protected hydroxyazetidine by treatment with a fluorinating agent to provide a protected fluoroazetidine;

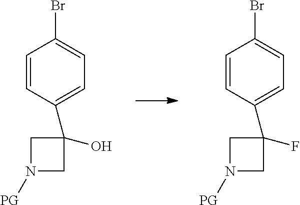

c) palladium catalyzed condensation of the bromophenylazetidine from steps a or b, above, with a vinyl ether to provide a protected 1-(4-(azetidin-3-yl)phenyl)ethanone derivative, wherein R² is hydroxyl or fluoro and R⁴ is a C₁-C₆alkyl;

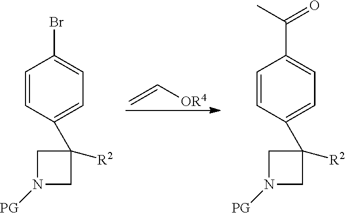

d) condensation of the 1-(4-(azetidin-3-yl)phenyl)ethanone derivative with a substituted trifluorophenylethanone to provide a 1-(4-(azetidin-3-yl)phenyl)-4,4,4-trifluoro-3-phenylbut-2-en-1-one analog;

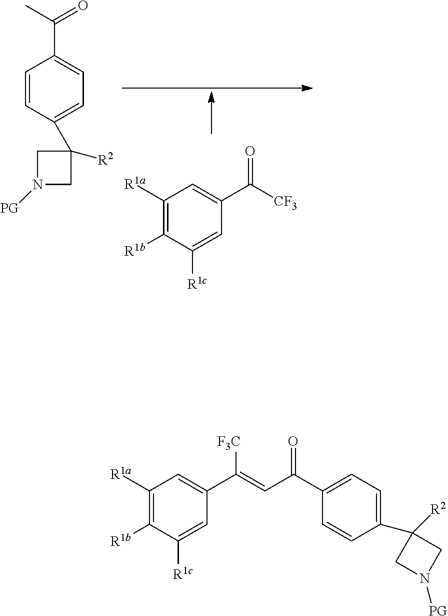

e) addition of hydroxylamine to the 1-(4-(azetidin-3-yl)phenyl)-4,4,4-trifluoro-3-phenylbut-2-en-1-one analog and cyclization in the presence of a quinine based chiral catalyst to provide a protected 3-(4-(azetidin-3-yl)phenyl)-5-phenyl-5-(trifluoromethyl)-4,5-dihydroisoxazole analog, wherein * denotes a chiral center;

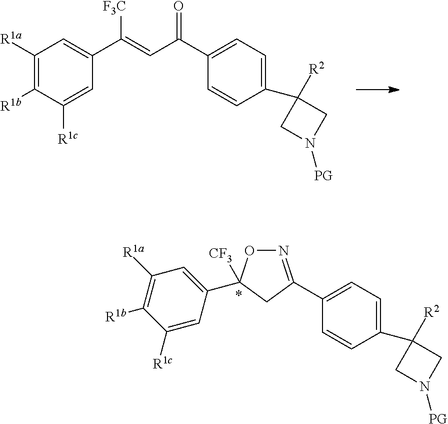

f) removal of the azetidine protecting group to provide a 3-(4-(azetidin-3-yl)phenyl)-5-phenyl-5-(trifluoromethyl)-4,5-dihydroisoxazole analog; and

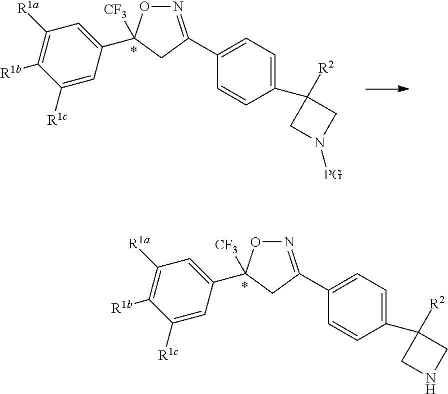

g) Coupling the 3-(4-(azetidin-3-yl)phenyl)-5-phenyl-5-(trifluoromethyl)-4,5-dihydroisoxazole analog with an acid or acid chloride under standard amide formation conditions.

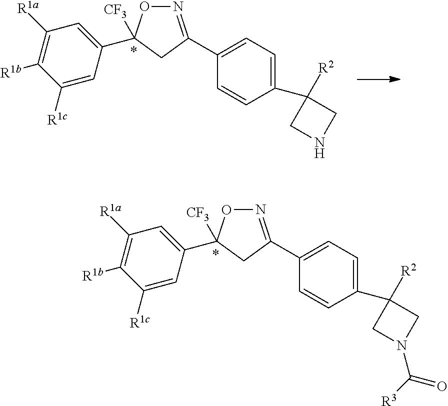

In another aspect of the invention, R^{1a}, R^{1b}, and R^{1c} are each independently hydrogen, chloro, fluoro, bromo, or C₁-C₆haloalkyl. In yet another aspect of the invention, R^{1a}, R^{1b}, and R^{1c} are each independently hydrogen, chloro, fluoro, bromo, or trifluoromethyl. In yet another aspect of the invention, R^{1a} and R^{1c} are each chloro, and R^{1b} is fluoro, chloro or hydrogen. In yet another aspect of the invention, R^{1a} and R^{1c} are each chloro, and R^{1b} is fluoro. In yet another aspect of the invention, R^{1a} and R^{1c} are each chloro, and R^{1b} is chloro. In yet another aspect of the invention, R^{1a} and R^{1c} are each chloro, and R^{1b} is hydrogen.

In another aspect of the invention, R² is fluoro. In yet another aspect of the invention, R² is hydroxyl.

In another aspect of the invention, R³ is C₁-C₆alkyl, C₀-C₆alkylC₃-C₆cycloalkyl, C₀-C₆alkylheteroaryl, or C₀-C₆alkylheterocycle. In yet another aspect of the invention, R³ is methyl, ethyl, propyl, isopropyl, butyl, isobutyl, t-butyl, cyclopropyl, or cyclobutyl, wherein each substituent can be optionally and independently substituted by at least one substituent selected from halo, hydroxyl, C₁-C₆haloalkyl, or —S(O)ₙR^{c}; and the cyclopropyl and cyclobutyl can further be optionally substituted with C₁-C₆alkyl or hydroxyC₁-C₆alkyl-; or R³ is thietanyl, thietanyl-1-oxide, thietanyl-1,1-dioxide, pyrazolyl, -CH₂-pyridyl or —CH₂pyrazolyl, wherein each substituent can be further optionally substituted with at least one substituent selected from halo, or C₁-C₆alkyl; and R^{c} is C₁-0₄ alkyl. In another aspect of the invention, R³ is —CH₂S(O)₂CH₃ or thietan-3-yl-1,1-dioxide.

In another aspect of the invention, the chiral quinine-based catalyst is selected from:

(2S)-1-(acridin-9-ylmethyl)-2-((R)-hydroxy(6-methoxyquinolin-4-yl)methyl)-5-vinylquinuclidin-1-ium bromide;

(2S)-1-(acridin-9-ylmethyl)-2-((R)-hydroxy(6-methoxyquinolin-4-yl)methyl)-5-vinylquinuclidin-1-ium chloride;

(2S)-1-(anthracen-9-ylmethyl)-2-((R)-hydroxy(6-methoxyquinolin-4-yl)methyl)-5-vinylquinuclidin-1-ium bromide; and

(2S)-1-(anthracen-9-ylmethyl)-2-((R)-hydroxy(6-methoxyquinolin-4-yl)methyl)-5-vinylquinuclidin-1-ium chloride.

In another aspect of the invention, the chiral quinine-based catalyst is selected from:

(2S)-1-(acridin-9-ylmethyl)-2-((R)-hydroxy(6-methoxyquinolin-4-yl)methyl)-5-vinylquinuclidin-1-ium bromide or (2S)-1-(acridin-9-ylmethyl)-2-((R)-hydroxy(6-methoxyquinolin-4-yl)methyl)-5-vinylquinuclidin-1-ium chloride.

In another aspect of the invention, the chiral quinine-based catalyst is

(2S)-1-(acridin-9-ylmethyl)-2-((R)-hydroxy(6-methoxyquinolin-4-yl)methyl)-5-vinylquinuclidin-1-ium bromide.

In another aspect of the invention, are compounds selected from:

tert-butyl 3-(4-bromophenyl)-3-hydroxyazetidine-1-carboxylate;

benzyl 3-(4-bromophenyl)-3-hydroxyazetidine-1-carboxylate;

1-benzhydryl-3-(4-bromophenyl)azetidin-3-ol;

tert-butyl 3-(4-bromophenyl)-3-fluoroazetidine-1-carboxylate;

benzyl 3-(4-bromophenyl)-3-fluoroazetidine-1-carboxylate;

1-benzhydryl-3-(4-bromophenyl)-3-fluoroazetidine;

tert-butyl 3-(4-acetylphenyl)-3-hydroxyazetidine-1-carboxylate;

benzyl 3-(4-acetylphenyl)-3-hydroxyazetidine-1-carboxylate;

1-(4-(1-benzhydryl-3-hydroxyazetidin-3-yl)phenyl)ethanone;

tert-butyl 3-(4-acetylphenyl)-3-fluoroazetidine-1-carboxylate;

benzyl 3-(4-acetylphenyl)-3-fluoroazetidine-1-carboxylate;

1-(4-(1-benzhydryl-3-fluoroazetidin-3-yl)phenyl)ethanone;

(E/Z)-tert-butyl 3-(4-(3-(3,5-dichloro-4-fluorophenyl)-4,4,4-trifluorobut-2-enoyl)phenyl)-3-hydroxyazetidine-1-carboxylate;

(E/Z)-tert-butyl 3-hydroxy-3-(4-(4,4,4-trifluoro-3-(3,4,5-trichlorophenyl)but-2-enoyl)phenyl)azetidine-1-carboxylate;

(E/Z)-tert-butyl 3-(4-(3-(3,5-dichlorophenyl)-4,4,4-trifluorobut-2-enoyl)phenyl)-3-hydroxyazetidine-1-carboxylate;

(E/Z)-tert-butyl 3-(4-(3-(3,4-dichloro-5-(trifluoromethyl)phenyl)-4,4,4-trifluorobut-2-enoyl)phenyl)-3-hydroxyazetidine-1-carboxylate;

(E/Z)-benzyl 3-(4-(3-(3, 5-dichloro-4-fluorophenyl)-4,4,4-trifluorobut-2-enoyl)phenyl)-3-hydroxyazetidine-1-carboxylate;

(E/Z)-benzyl 3-hydroxy-3-(4-(4,4,4-trifluoro-3-(3,4,5-trichlorophenyl)but-2-enoyl)phenyl)azetidine-1-carboxylate;

(E/Z)-benzyl 3-(4-(3-(3,5-dichlorophenyl)-4,4,4-trifluorobut-2-enoyl)phenyl)-3-hydroxyazetidine-1-carboxylate;

(E/Z)-benzyl 3-(4-(3-(3,4-dichloro-5-(trifluoromethyl)phenyl)-4,4,4-trifluorobut-2-enoyl)phenyl)-3-hydroxyazetidine-1-carboxylate;

(E/Z)-1-(4-(1-benzhydryl-3-hydroxyazetidin-3-yl)phenyl)-3-(3,5-dichloro-4-fluorophenyl)-4,4,4-trifluorobut-2-en-1-one ;

(E/Z)-1-(4-(1-benzhydryl-3-hydroxyazetidin-3-yl)phenyl)-3-(3,5-dichlorophenyl)-4,4,4-trifluorobut-2-en-1-one ;

(E/Z)-1-(4-(1-benzhydryl-3-hydroxyazetidin-3-yl)phenyl)-4,4,4-trifluoro-3-(3,4,5-trichlorophenyl)but-2-en-1-one;

(E/Z)-1-(4-(1-benzhydryl-3-hydroxyazetidin-3-yl)phenyl)-3-(3,4-dichloro-5-(trifluoromethyl)phenyl)-4,4,4-trifluorobut-2-en-1-one;

(E/Z)-tert-butyl 3-(4-(3-(3,5-dichloro-4-fluorophenyl)-4,4,4-trifluorobut-2-enoyl)phenyl)-3-fluoroazetidine-1-carboxylate;

(E/Z)-tert-butyl 3-fluoro-3-(4-(4,4,4-trifluoro-3-(3,4,5-trich lorophenyl)but-2-enoyl)phenyl)azetidine-1-carboxylate;

(E/Z)-tert-butyl 3-(4-(3-(3,5-dichlorophenyl)-4,4,4-trifluorobut-2-enoyl)phenyl)-3-fluoroazetidine-1-carboxylate;

(E/Z)-tert-butyl 3-(4-(3-(3,4-dichloro-5-(trifluoromethyl)phenyl)-4,4,4-trifluorobut-2-enoyl)phenyl)-3-fluoroazetidine-1-carboxylate;

(E/Z)-benzyl 3-(4-(3-(3,5-dichloro-4-fluorophenyl)-4,4,4-trifluorobut-2-enoyl)phenyl)-3-fluoroazetidine-1-carboxylate;

(E/Z)-benzyl 3-fluoro-3-(4-(4,4,4-trifluoro-3-(3,4, 5-trichlorophenyl)but-2-enoyl)phenyl)azetidine-1-carboxylate;

(E/Z)-benzyl 3-(4-(3-(3,5-dichlorophenyl)-4,4,4-trifluorobut-2-enoyl)phenyl)-3-fluoroazetidine-1-carboxylate;

(E/Z)-benzyl 3-(4-(3-(3,4-dichloro-5-(trifluoromethyl)phenyl)-4,4,4-trifluorobut-2-enoyl)phenyl)-3-fluoroazetidine-1-carboxylate;

(E/Z)-1-(4-(1-benzhydryl-3-fluoroazetidin-3-yl)phenyl)-3-(3,5-dichloro-4-fluorophenyl)-4,4,4-trifluorobut-2-en-1-one;

(E/Z)-1-(4-(1-benzhydryl-3-fluoroazetidin-3-yl)phenyl)-3-(3,5-dichlorophenyl)-4,4,4-trifluorobut-2-en-1-one;

(E/Z)-1-(4-(1-benzhydryl-3-fluoroazetidin-3-yl)phenyl)-4,4,4-trifluoro-3-(3,4,5-trichlorophenyl)but-2-en-1-one;

(E/Z)-1-(4-(1-benzhydryl-3-fluoroazetidin-3-yl)phenyl)-3-(3,4-dichloro-5-(trifluoromethyl)phenyl)-4,4,4-trifluorobut-2-en-1-one;

(S)-tert-butyl 3-(4-(5-(3,5-dichloro-4-fluorophenyl)-5-(trifluoromethyl)-4,5-dihydroisoxazol-3-yl)phenyl)-3-hydroxyazetidine-1-carboxylate;

(S)-tert-butyl 3-(4-(5-(3,5-dichlorophenyl)-5-(trifluoromethyl)-4,5-dihydroisoxazol-3-yl)phenyl)-3-hydroxyazetidine-1-carboxylate;

(S)-tert-butyl 3-hydroxy-3-(4-(5-(3,4,5-trichlorophenyl)-5-(trifluoromethyl)-4,5-dihydroisoxazol-3-yl)phenyl)azetidine-1-carboxylate;

(S)-tert-butyl 3-(4-(5-(3,4-dichloro-5-(trifluoromethyl)phenyl)-5-(trifluoromethyl)-4,5-dihydroisoxazol-3-yl)phenyl)-3-hydroxyazetidine-1-carboxylate;

(R)-tert-butyl 3-(4-(5-(3,5-dichloro-4-fluorophenyl)-5-(trifluoromethyl)-4,5-dihydroisoxazol-3-yl)phenyl)-3-hydroxyazetidine-1-carboxylate;

(R)-tert-butyl 3-(4-(5-(3,5-dichlorophenyl)-5-(trifluoromethyl)-4,5-dihydroisoxazol-3-yl)phenyl)-3-hydroxyazetidine-1-carboxylate;

(R)-tert-butyl 3-hydroxy-3-(4-(5-(3,4,5-trichlorophenyl)-5-(trifluoromethyl)-4,5-dihydroisoxazol-3-yl)phenyl)azetidine-1-carboxylate;

(R)-tert-butyl 3-(4-(5-(3,4-dichloro-5-(trifluoromethyl)phenyl)-5-(trifluoromethyl)-4,5-dihydroisoxazol-3-yl)phenyl)-3-hydroxyazetidine-1-carboxylate;

(S)-tert-butyl 3-(4-(5-(3,5-dichloro-4-fluorophenyl)-5-(trifluoromethyl)-4,5-dihydroisoxazol-3-yl)phenyl)-3-fluoroazetidine-1-carboxylate;

(S)-tert-butyl 3-(4-(5-(3,5-dichlorophenyl)-5-(trifluoromethyl)-4,5-dihydroisoxazol-3-yl)phenyl)-3-fluoroazetidine-1-carboxylate;

(S)-tert-butyl 3-fluoro-3-(4-(5-(3,4,5-trichlorophenyl)-5-(trifluoromethyl)-4,5-dihydroisoxazol-3-yl)phenyl)azetidine-1-carboxylate;

(S)-tert-butyl 3-(4-(5-(3,4-dichloro-5-(trifluoromethyl)phenyl)-5-(trifluoromethyl)-4,5-dihydroisoxazol-3-yl)phenyl)-3-fluoroazetidine-1-carboxylate;

(R)-tert-butyl 3-(4-(5-(3,5-dichloro-4-fluorophenyl)-5-(trifluoromethyl)-4,5-dihydroisoxazol-3-yl)phenyl)-3-fluoroazetidine-1-carboxylate;

(R)-tert-butyl 3-(4-(5-(3,5-dichlorophenyl)-5-(trifluoromethyl)-4,5-dihydroisoxazol-3-yl)phenyl)-3-fluoroazetidine-1-carboxylate;

(R)-tert-butyl 3-fluoro-3-(4-(5-(3,4, 5-trichlorophenyl)-5-(trifluoromethyl)-4,5-dihydroisoxazol-3-yl)phenyl)azetidine-1-carboxylate;

(R)-tert-butyl 3-(4-(5-(3,4-dichloro-5-(trifluoromethyl)phenyl)-5-(trifluoromethyl)-4,5-dihydroisoxazol-3-yl)phenyl)-3-fluoroazetidine-1-carboxylate;

(S)-benzyl 3-(4-(5-(3,5-dichloro-4-fluorophenyl)-5-(trifluoromethyl)-4,5-dihydroisoxazol-3-yl)phenyl)-3-hydroxyazetidine-1-carboxylate;

(S)-benzyl 3-(4-(5-(3,5-dichlorophenyl)-5-(trifluoromethyl)-4,5-dihydroisoxazol-3-yl)phenyl)-3-hydroxyazetidine-1-carboxylate;

(S)-benzyl 3-hydroxy-3-(4-(5-(3,4,5-trichlorophenyl)-5-(trifluoromethyl)-4,5-dihydroisoxazol-3-yl)phenyl)azetidine-1-carboxylate;

(S)-benzyl 3-(4-(5-(3,4-dichloro-5-(trifluoromethyl)phenyl)-5-(trifluoromethyl)-4,5-dihydroisoxazol-3-yl)phenyl)-3-hydroxyazetidine-1-carboxylate;

(R)-benzyl 3-(4-(5-(3,5-dichloro-4-fluorophenyl)-5-(trifluoromethyl)-4,5-dihydroisoxazol-3-yl)phenyl)-3-hydroxyazetidine-1-carboxylate;

(R)-benzyl 3-(4-(5-(3,5-dichlorophenyl)-5-(trifluoromethyl)-4,5-dihydroisoxazol-3-yl)phenyl)-3-hydroxyazetidine-1-carboxylate;

(R)-benzyl 3-hydroxy-3-(4-(5-(3,4, 5-trichlorophenyl)-5-(trifluoromethyl)-4,5-dihydroisoxazol-3-yl)phenyl)azetidine-1-carboxylate;

(R)-benzyl 3-(4-(5-(3,4-dichloro-5-(trifluoromethyl)phenyl)-5-(trifluoromethyl)-4,5-dihydroisoxazol-3-yl)phenyl)-3-hydroxyazetidine-1-carboxylate;

(S)-benzyl 3-(4-(5-(3,5-dichloro-4-fluorophenyl)-5-(trifluoromethyl)-4,5-dihydroisoxazol-3-yl)phenyl)-3-fluoroazetidine-1-carboxylate;

(S)-benzyl 3-(4-(5-(3,5-dichlorophenyl)-5-(trifluoromethyl)-4,5-dihydroisoxazol-3-yl)phenyl)-3-fluoroazetidine-1-carboxylate;

(S)-benzyl 3-fluoro-3-(4-(5-(3,4, 5-trichlorophenyl)-5-(trifluoromethyl)-4, 5-dihydroisoxazol-3-yl)phenyl)azetidine-1-carboxylate;

(S)-benzyl 3-(4-(5-(3,4-dichloro-5-(trifluoromethyl)phenyl)-5-(trifluoromethyl)-4,5-dihydroisoxazol-3-yl)phenyl)-3-fluoroazetidine-1-carboxylate;

(R)-benzyl 3-(4-(5-(3,5-dichloro-4-fluorophenyl)-5-(trifluoromethyl)-4,5-dihydroisoxazol-3-yl)phenyl)-3-fluoroazetidine-1-carboxylate;

(R)-benzyl 3-(4-(5-(3,5-dichlorophenyl)-5-(trifluoromethyl)-4,5-dihydroisoxazol-3-yl)phenyl)-3-fluoroazetidine-1-carboxylate;

(R)-benzyl 3-fluoro-3-(4-(5-(3,4,5-trichlorophenyl)-5-(trifluoromethyl)-4,5-dihydroisoxazol-3-yl)phenyl)azetidine-1-carboxylate;

(R)-benzyl 3-(4-(5-(3,4-dichloro-5-(trifluoromethyl)phenyl)-5-(trifluoromethyl)-4,5-dihydroisoxazol-3-yl)phenyl)-3-fluoroazetidine-1-carboxylate;

(S)-3-(4-(1-benzhydryl-3-hydroxyazetidin-3-yl)phenyl)-5-(3,5-dichloro-4-fluorophenyl)-5-(trifluoromethyl)-4,5-dihydroisoxazole;

(S)-3-(4-(1-benzhydryl-3-hydroxyazetidin-3-yl)phenyl)-5-(3,5-dichlorophenyl)-5-(trifluoromethyl)-4,5-dihydroisoxazole;

(S)-3-(4-(1-benzhydryl-3-hydroxyazetidin-3-yl)phenyl)-5-(3,4,5-trichlorophenyl)-5-(trifluoromethyl)-4,5-dihydroisoxazole;

(S)-3-(4-(1-benzhydryl-3-hydroxyazetidin-3-yl)phenyl)-5-(3,4-dichloro-5-(trifluoromethyl)phenyl)-5-(trifluoromethyl)-4,5-dihydroisoxazole;

(R)-3-(4-(1-benzhydryl-3-hydroxyazetidin-3-yl)phenyl)-5-(3,5-dichloro-4-fluorophenyl)-5-(trifluoromethyl)-4,5-dihydroisoxazole;

(R)-3-(4-(1-benzhydryl-3-hydroxyazetidin-3-yl)phenyl)-5-(3,5-dichlorophenyl)-5-(trifluoromethyl)-4,5-dihydroisoxazole;

(R)-3-(4-(1-benzhydryl-3-hydroxyazetidin-3-yl)phenyl)-5-(3,4,5-trichlorophenyl)-5-(trifluoromethyl)-4,5-dihydroisoxazole;

(R)-3-(4-(1-benzhydryl-3-hydroxyazetidin-3-yl)phenyl)-5-(3,4-dichloro-5-(trifluoromethyl)phenyl)-5-(trifluoromethyl)-4,5-dihydroisoxazole;

(S)-3-(4-(1-benzhydryl-3-fluoroazetidin-3-yl)phenyl)-5-(3,5-dichloro-4-fluorophenyl)-5-(trifluoromethyl)-4,5-dihydroisoxazole;

(S)-3-(4-(1-benzhydryl-3-fluoroazetidin-3-yl)phenyl)-5-(3,5-dichlorophenyl)-5-(trifluoromethyl)-4,5-dihydroisoxazole;

(S)-3-(4-(1-benzhydryl-3-fluoroazetidin-3-yl)phenyl)-5-(3,4,5-trichlorophenyl)-5-(trifluoromethyl)-4,5-dihydroisoxazole;

(S)-3-(4-(1-benzhydryl-3-fluoroazetidin-3-yl)phenyl)-5-(3,4-dichloro-5-(trifluoromethyl)phenyl)-5-(trifluoromethyl)-4,5-dihydroisoxazole;

(R)-3-(4-(1-benzhydryl-3-fluoroazetidin-3-yl)phenyl)-5-(3,5-dichloro-4-fluorophenyl)-5-(trifluoromethyl)-4,5-dihydroisoxazole;

(R)-3-(4-(1-benzhydryl-3-fluoroazetidin-3-yl)phenyl)-5-(3,5-dichlorophenyl)-5-(trifluoromethyl)-4,5-dihydroisoxazole;

(R)-3-(4-(1-benzhydryl-3-fluoroazetidin-3-yl)phenyl)-5-(3,4,5-trichlorophenyl)-5-(trifluoromethyl)-4,5-dihydroisoxazole;

(R)-3-(4-(1-benzhydryl-3-fluoroazetidin-3-yl)phenyl)-5-(3,4-dichloro-5-(trifluoromethyl)phenyl)-5-(trifluoromethyl)-4,5-dihydroisoxazole;

(S)-3-(4-(5-(3, 5-dichloro-4-fluorophenyl)-5-(trifluoromethyl)-4,5-dihydroisoxazol-3-yl)phenyl)azetidin-3-ol;

(S)-3-(4-(5-(3,5-dichlorophenyl)-5-(trifluoromethyl)-4,5-dihydroisoxazol-3-yl)phenyl)azetidin-3-ol;

(S)-3-(4-(5-(3,4,5-trichlorophenyl)-5-(trifluoromethyl)-4,5-dihydroisoxazol-3-yl)phenyl)azetidin-3-ol;

(S)-3-(4-(5-(3,4-dichloro-5-(trifluoromethyl)phenyl)-5-(trifluoromethyl)-4,5-dihydroisoxazol-3-yl)phenyl)azetidin-3-ol;

(R)-3-(4-(5-(3,5-dichloro-4-fluorophenyl)-5-(trifluoromethyl)-4,5-dihydroisoxazol-3-yl)phenyl)azetidin-3-ol;

(R)-3-(4-(5-(3,5-dichlorophenyl)-5-(trifluoromethyl)-4,5-dihydroisoxazol-3-yl)phenyl)azetidin-3-ol;

(R)-3-(4-(5-(3,4,5-trichlorophenyl)-5-(trifluoromethyl)-4,5-dihydroisoxazol-3-yl)phenyl)azetidin-3-ol;

(R)-3-(4-(5-(3,4-dichloro-5-(trifluoromethyl)phenyl)-5-(trifluoromethyl)-4,5-dihydroisoxazol-3-yl)phenyl)azetidin-3-ol;

(S)-5-(3,5-dichloro-4-fluorophenyl)-3-(4-(3-fluoroazetidin-3-yl)phenyl)-5-(trifluoromethyl)-4,5-dihydroisoxazole;

(S)-5-(3,5-dichlorophenyl)-3-(4-(3-fluoroazetidin-3-yl)phenyl)-5-(trifluoromethyl)-4,5-dihydroisoxazole;

(S)-3-(4-(3-fluoroazetidin-3-yl)phenyl)-5-(3,4,5-trichlorophenyl)-5-(trifluoromethyl)-4,5-dihydroisoxazole;

(S)-5-(3,4-dichloro-5-(trifluoromethyl)phenyl)-3-(4-(3-fluoroazetidin-3-yl)phenyl)-5-(trifluoromethyl)-4,5-dihydroisoxazole;

(R)-5-(3,5-dichloro-4-fluorophenyl)-3-(4-(3-fluoroazetidin-3-yl)phenyl)-5-(trifluoromethyl)-4,5-dihydroisoxazole;

(R)-5-(3,5-dichlorophenyl)-3-(4-(3-fluoroazetidin-3-yl)phenyl)-5-(trifluoromethyl)-4,5-dihydroisoxazole;

(R)-3-(4-(3-fluoroazetidin-3-yl)phenyl)-5-(3,4,5-trichlorophenyl)-5-(trifluoromethyl)-4,5-dihydroisoxazole; and

(R)-5-(3,4-dichloro-5-(trifluoromethyl)phenyl)-3-(4-(3-fluoroazetidin-3-yl)phenyl)-5-(trifluoromethyl)-4,5-dihydroisoxazole.

In another aspect of the invention are compounds selected from:

3-(4-acetyl-phenyl)-3-fluoro-azetidine-1-carboxylic acid tert-butyl ester;

(Z)-tert-butyl 3-(4-(3-(3,5-dichloro-phenyl)-4,4,4-trifluoro-but-2-enoyl)phenyl)-3-fluoroazetidine-1-carboxylate;

(Z)-tert-butyl 3-(4-(3-(3,5-dichloro-4-fluoro-phenyl)-4,4,4-trifluoro-but-2-enoyl)-phenyl)-3-fluoro-azetidine-1-carboxylate;

(Z)-tert-butyl 3-(4-(3-(3,4,5-trichloro-phenyl)-4,4,4-trifluoro-but-2-enoyl)-phenyl)-3-fluoro-azetidine-1-carboxylate; and

(R)-tert-butyl 3-(4-(5-(3,5-dichloro-4-fluorophenyl)-5-(trifluoromethyl)-4,5-dihydroisoxazol-3-yl)phenyl)-3-fluoroazetidine-1-carboxylate.

In another aspect of the invention are compounds selected from:

(Z)-tert-butyl 3-(4-(3-(3,5-dichloro-phenyl)-4,4,4-trifluoro-but-2-enoyl)phenyl)-3-fluoroazetidine-1-carboxylate;

(Z)-tert-butyl 3-(4-(3-(3,5-dichloro-4-fluoro-phenyl)-4,4,4-trifluoro-but-2-enoyl)-phenyl)-3-fluoro-azetidine-1-carboxylate; and

(Z)-tert-butyl 3-(4-(3-(3,4,5-trichloro-phenyl)-4,4,4-trifluoro-but-2-enoyl)-phenyl)-3-fluoro-azetidine-1-carboxylate.

In another aspect of the invention is the compound (R)-tert-butyl 3-(4-(5-(3,5-dichloro-4-fluorophenyl)-5-(trifluoromethyl)-4,5-dihydroisoxazol-3-yl)phenyl)-3-fluoroazetidine-1-carboxylate.

### DEFINITIONS

For purposes of the present invention, as described and claimed herein, the following terms and phrases are defined as follows:

“Additional veterinary agent(s)” as used herein, unless otherwise indicated, refers to other veterinary or pharmaceutical compounds or products that provide a therapeutically effective amount of said agent(s) that are useful for the treatment of a parasitic infection in an animal, as described herein.

“Alkoxy”, as used herein, unless otherwise indicated, refers to an oxygen moiety having a further alkyl substituent. The alkyl portion (i.e., alkyl moiety) of an alkoxy group has the same definition as below. Non-limiting examples include: —OCH₃, —OCH₂CH₃, and the like. Further when used in compound words such as haloalkoxy, said alkoxy moiety has the same meaning as herein defined and may be attached to the chemical moiety by any one of the carbon atoms of the aliphatic chain. Non-limiting examples of the compound word, haloalkoxy include —OCH₂F, —OCHF₂, —OCH₂CH₂F, —OCH₂Cl, —OCH₂CH₂Cl and the like.

“Alkyl”, as used herein, unless otherwise indicated, refers to saturated monovalent hydrocarbon alkane radicals of the general formula CₙH₂ₙ₊₁. The alkane radical may be straight or branched and may be unsubstituted or substituted. For example, the term “(C₁-C₆) alkyl” refers to a monovalent, straight or branched aliphatic group containing 1 to 6 carbon atoms. Non-exclusive examples of (C₁-C₆) alkyl groups include, but are not limited to methyl, ethyl, propyl, isopropyl, sec-butyl, t-butyl, n-propyl, n-butyl, i-butyl, s-butyl, n-pentyl, 1-methylbutyl, 2-methylbutyl, 3-methylbutyl, neopentyl, 3,3-dimethylpropyl, 2-methylpentyl, hexyl, and the like. The alkyl moiety may be attached to the chemical moiety by any one of the carbon atoms of the aliphatic chain. Alkyl groups are optionally substituted as described herein. Further when used in compound words such as alkylphenyl, said alkyl moiety has the same meaning as herein defined and may be attached to the chemical moiety by any one of the carbon atoms of the aliphatic chain. Non-limiting examples of the compound word, alkylphenyl include: C₁alkylphenyl is —CH₂phenyl, C₂alkylphenyl is —CH₂CH₂phenyl, C₀alkylphenyl is phenyl, and the like. Similarly, when used in a compound word like hydroxyalkyl, non-limiting examples include —CH₂OH, —CH₂CH₂OH, and the like.

“Alkenyl” as used herein, unless otherwise indicated, refers to a straight or branched aliphatic hydrocarbon chain having 2- to 6-carbon atoms and containing at least one carbon-carbon double bond (for example —C═C—, or —C═CH₂). Non-exclusive examples of alkenyl include: ethenyl, 1-propenyl, 2-propenyl, isopropenyl, 1-butenyl, 2-butenyl, 3-butenyl, 2-pentenyl, and the like.

“Alkynyl” as used herein, unless otherwise indicated, refers to straight or branched aliphatic hydrocarbon chain having 2- to 6-carbon atoms and containing at least one carbon-carbon triple bond (for example, —C≡C— or —C≡CH). Non- exclusive examples of alkynyl include: ethynyl, 2-propynyl, 1-methyl-2-propynyl, 2-butynyl, 3-butynyl, 2-methyl-3-butynyl, and the like.

“Animal(s)”, as used herein, unless otherwise indicated, refers to an individual animal that is a mammal, bird, or fish. Specifically, mammal refers to a vertebrate animal that is human and non-human, which are members of the taxonomic class Mammalia. Non-exclusive examples of non-human mammals include companion animals and livestock. Non-exclusive examples of a companion animal include: dog, cat, llama, and horse. Preferred companion animals are dog, cat, and horse. More preferred is dog. Non-exclusive examples of livestock include: swine, camel, rabbit, goat, sheep, deer, elk, cattle, and bison. Preferred livestock is cattle and swine. Specifically, bird refers to a vertebrate animal of the taxonomic class Ayes. Birds are feathered, winged, bipedal, endothermic, and egg-laying. Non-exclusive examples of bird include, poultry (e.g., chicken, turkey, duck, and geese), all of which are also referred to herein as fowl. Specifically, fish refers to the taxonomic class Chondrichthyes (cartilaginous fishes, e.g., sharks and rays) and Osteichthyes (bony fishes) which live in water, have gills or mucus-covered skin for respiration, fins, and may have scales. Non-exclusive examples of fish include shark, salmon, trout, whitefish, catfish, tilapia, sea bass, tuna, halibut, turbot, flounder, sole, striped bass, eel, yellowtail, grouper, and the like.

“Carbocyclic”, as used herein, unless otherwise indicated, refers to a partially saturated or saturated 5- to 7-membered ring containing only carbon atoms and can be monocyclic or part of a fused ring or spiro ring moiety. Examples of carbocyclic rings include cyclopentane, cyclohexane, and cycloheptane. The carbocyclic ring is optionally substituted as described herein.

“Chiral”, as used herein, unless otherwise indicated, refers to the structural characteristic of a molecule that makes it impossible to superimpose it on its mirror image, and includes both the “R” and “S” designations of the compound.

“Compounds of the present invention”, as used herein, unless otherwise indicated, refers to compounds of Formula (1), and stereoisomers thereof.

“Cycloalkyl”, as used herein, unless otherwise indicated, includes fully saturated or partially saturated carbocyclic alkyl moieties. Non-limiting examples of partially saturated cycloalkyls include: cyclopropene, cyclobutene, cycloheptene, cyclooctene, cyclohepta-1,3-diene, and the like. Preferred cycloalkyls are 3- to 6-membered saturated monocyclic rings including cyclopropyl, cyclobutyl, cyclopentyl, and cyclohexyl. The cycloalkyl group may be attached to the chemical moiety by any one of the carbon atoms within the carbocyclic ring. Cycloalkyl groups are optionally substituted with at least one substituent. Further when used in compound words such as alkylcycloalkyl, said alkyl and cycloalkyl moiety has the same meaning as herein defined and may be attached to the chemical moiety by any one of the carbon atoms of the aliphatic chain. Examples of “alkylcycloalkyl” include, methylcyclopropane (C₁alkylC₃cycloalkyl or —CH₂cyclopropane), ethylcyclopropane (C₂alkylC₃cycloalkyl or —CH₂CH₂cyclopropane), methylcyclobutane (C₁alkylC₄cycloalkyl or —CH₂cyclobutane), ethylcyclobutane (C₂alkylC₄cycloalkyl or —CH₂CH₂cyclobutane), methylcyclohexane (C₁alkylC₆cycloalkyl or —CH₂cyclohexane), and the like. C₀alkylC₃-C₆cycloalkyl is C₃-C₆cycloalkyl. Cycloalkyl moieties are optionally substituted as described herein.

“Enantiomer (enantiomerically) enriched”, as used herein, unless otherwise indicated, refers to a mixture of enantiomers wherein one of the “S” or “R” enantiomers constitutes at least 65% of the combined mixture.

“Enantiomer (enantiomerically) pure”, as used herein, unless otherwise indicated, refers to a mixture of enantiomers wherein one of the “S” or “R” enantiomers constitutes at least 95% of the combined mixture.

“Halogen” or “halo”, as used herein, unless otherwise indicated, refers to fluorine, chlorine, bromine and iodine. Further, when used in compound words such as “haloalkyl”, “haloalkoxy”, “haloalkenyl”, or “haloalkynyl”, said alkyl, alkoxy, alkenyl, and alkynyl may be partially or fully substituted with halogen atoms which may be the same or different and said alkyl, alkoxy, alkenyl, and alkynyl moiety has the same meaning as above and may be attached to the chemical moiety by any one of the carbon atoms of the aliphatic chain. Examples of “haloalkyl” include F₃C—, ClCH₂—, CF₃CH₂— and CF₃CCl₂—, and the like. The term “haloalkoxy” is defined analogously to the term “haloalkyl”. Examples of “haloalkoxy” include CF₃O—, CCl₃CH₂O—, HCF₂CH₂CH₂O— and CF₃CH₂O—, and the like. The term “haloalkenyl is defined analogously to the term “haloalkyl” except that the aliphatic chain contains at least one carbon-carbon double bond. Examples of “haloalkenyl” include CF₃C═C—, CCl₃C═C—, HCF₂C═C— and CF₃C=CC—, and the like. The term “haloalkynyl” is defined analogously to the term “haloalkyl” except that the aliphatic chain contains at least one carbon-carbon triple bond. Examples of “haloalkynyl” include CF₃C≡C—, CCl₃C≡C—, HCF₂C≡C— and CF₃C≡CC—, and the like.

“Heteroaryl” or “Het”, as used herein, unless otherwise indicated, refers to a 5- to 6-membered aromatic monocyclic ring or an 8- to 10-membered fused aromatic ring where said monocyclic- and fused-ring moiety contains one or more heteroatoms each independently selected from N, O, or S, preferably from one to four heteroatoms. Non-exclusive examples of monocyclic heteroaryls include pyrrolyl, furanyl, thiophenyl, pyrazolyl, imidazolyl, triazolyl, tetrazolyl, thiazolyl, isoxazolyl, oxazolyl, oxadiazolyl, thiadiazolyl, pyridinyl, pyridazinyl, pyrimidinyl, pyrazinyl, and the like. Non-exclusive examples of fused heteroaryls include: benzofuranyl, benzothiophenyl, indolyl, benzimidazolyl, indazolyl, benzotriazolyl, thieno[2,3-c]pyridine, thieno[3,2-b]pyridine, benzo[1,2,5]thiadiazole, and the like. The heteroaryl group may be attached to the chemical moiety by any one of the carbon atoms or heteroatoms (e.g., N, O, and S) within the monocyclic or fused ring. Further when used in compound words such as alkylheteroaryl, said alkyl and heteroaryl moiety have the same meaning as herein defined and may be attached to the chemical moiety by any one of the carbon atoms of the aliphatic chain. For example, C₀alkylheteroaryl is heteroaryl, C₁alkylheteroaryl is —CH₂heteroaryl, C₀alkylheteroaryl is —CH₂CH₂heteroaryl, and the like. Heteroaryls are optionally substituted as described herein.

“Heterocycle”, as used herein, unless otherwise indicated, refers to a partially saturated or saturated 3- to 7-membered monocyclic ring containing one or more heteroatoms each independently selected from N, O, or S, preferably from one to four heteroatoms. The heterocyclic ring can be part of a fused ring or spiro-ring moiety. Non-exclusive examples of heterocycle include oxirane, thiarane, aziridine, oxetane, azetidine, thiatane, tetrahydrofuran, tetrahydrothiophene, pyrrolidine, tetrahydropyrane, piperidine, piperazine, tetrahydropyridine, 2H-azirine, 2,3-dihydro-azete, 3,4-dihydro-2H-pyrrole, and the like. The heterocycle group may be attached to the chemical moiety by any one of the carbon atoms or heteroatoms (e.g., N, O, and S) within the ring. Further when used in compound words such as alkylheterocycle, said alkyl and heterocycle moiety have the same meaning as herein defined and may be attached to the chemical moiety by any one of the carbon atoms of the aliphatic chain. For example, C₀alkylheterocycle is heterocycle, C₁alkylheterocycle is —CH₂heterocycle, C₀alkylheterocycle is —CH₂CH₂heterocycle, and the like. Heterocycles are optionally substituted as described herein.

“Optionally substituted”, is used herein interchangeably with the phrase substituted or unsubstituted. Unless otherwise indicated, an optionally substituted group may have a substituent at each substitutable position of the group, and each substitution is independent of the other. An optionally substituted group also may have no substituents. Therefore, the phrase “optionally substituted with at least one substituent” means that the number of substituents may vary from zero up to a number of available positions for substitution.

“Parasite(s)”, as used herein, unless otherwise indicated, refers to endoparasites and ectoparasites. Endoparasites are parasites that live within the body of its host and include helminths (e.g., trematodes, cestodes, and nematodes) and protozoa. Ectoparasites are organisms of the Arthropoda phylum (e.g., arachnids, insects, and crustaceans (e.g., copepods-sea lice) which feed through or upon the skin of its host. Preferred arachnids are of the order Acarina, e.g., ticks and mites. Preferred insects are midges, fleas, mosquitos, biting flies (stable fly, horn fly, blow fly, horse fly, and the like) and lice. Preferred compounds of the present invention can be used for the treatment of parasites, i.e., treatment of a parasitic infection or infestation.

“Protecting group” or “PG”, as used herein, unless otherwise indicated, refers to a substituent that is commonly employed to block or protect an amine on the compound thereby protecting its functionality while allowing for the reaction of other functional groups on the compound. Non-exclusive examples of an amine-protecting group include: acyl groups (e.g., formyl, acetyl, chloroacetyl, trichloro-acetyl, o-nitrophenylacetyl, o-nitrophenoxyacetyl, trifluoroacetyl, acetoacetyl, 4-chlorobutyryl, isobutyryl, o-nitrocinnamoyl, picolinoyl, acylisothiocyanate, aminocaproyl, benzoyl, and the like), acyloxy groups (e.g., 1-tert-butyloxycarbonyl (Boc), methoxycarbonyl, 9-fluorenyl-methoxycarbonyl, 2,2,2-trifluoroethoxycarbonyl, 2-trimethylsilylethxoycarbonyl, vinyloxycarbonyl, allyloxycarbonyl, 1,1-dimethyl-propynyloxycarbonyl, benzyloxy-carbonyl, p-nitrobenzyloxycarbony, 2,4-dichlorobenzyloxycarbonyl, and the like), diphenylmethane, and benzylcarbamates.

“Therapeutically effective amount”, as used herein, unless otherwise indicated, refers to an amount of the compounds of the present invention that (i) treat the particular parasitic infection or infestation, (ii) attenuates, ameliorates, or eliminates one or more symptoms of the particular parasitic infection or infestation, or (iii) prevents or delays the onset of one or more symptoms of the particular parasitic infection or infestation described herein.

“Treatment”, “treating”, and the like, as used herein, unless otherwise indicated, refers to reversing, alleviating, or inhibiting the parasitic infection, infestation, or condition. As used herein, these terms also encompass, depending on the condition of the animal, preventing the onset of a disorder or condition, or of symptoms associated with a disorder or condition, including reducing the severity of a disorder or condition or symptoms associated therewith prior to affliction with said infection or infestation. Thus, treatment can refer to administration of the compounds of the present invention to an animal that is not at the time of administration afflicted with the infection or infestation. Treating also encompasses preventing the recurrence of an infection or infestation or of symptoms associated therewith as well as references to “control” (e.g., kill, repel, expel, incapacitate, deter, eliminate, alleviate, minimize, and eradicate).

“Veterinary acceptable” as used herein, unless otherwise indicated, indicates that the substance or composition must be compatible chemically and/or toxicologically, with the other ingredients comprising a formulation, composition, and/or the animal being treated therewith. The term “pharmaceutically” acceptable has the same meaning as that recited for “veterinarily” acceptable, and therefore can be used interchangeably.

### DETAILED DESCRIPTION

The present invention provides a process for the preparation of Formula (1) compounds, stereoisomers thereof, as well as veterinary compositions that are useful as antiparasitic agents for animals, in particular, compounds that act as ectoparasiticides.

Compounds of the present invention may be synthesized by synthetic routes that include processes analogous to those well known in the chemical arts, particularly in light of the description contained herein. The starting materials are generally available from commercial sources such as Aldrich Chemicals (Milwaukee, Wis.) or are readily prepared using methods well known to those skilled in the art (e.g., prepared by methods generally described in Louis F. Fieser and Mary Fieser, “Reagents for Organic Synthesis”, 1; 19, Wiley, New York (1967, 1999 ed.), or Beilsteins Handbuch der organischen Chemie, 4, Aufl. ed. Springer-Verlag, Berlin, including supplements (also available via the Beilstein online database)). For illustrative purposes, the reaction schemes depicted below demonstrate potential routes for synthesizing compounds of the present invention, and key intermediates. For a more detailed description of the individual reaction steps, see the Examples section below. A skilled artisan will appreciate that other suitable starting materials, reagents, and synthetic routes may be used to synthesize the compounds of the present invention and a variety of derivatives thereof. Further, many of the compounds prepared by the methods described below can be further modified in light of this disclosure using conventional chemistry well known to the skilled artisan.

Compounds of the present invention described herein contain at least one asymmetric or chiral center; and, therefore, exist in different stereoisomeric forms. The R and S configurations are based upon knowledge of known chiral inversion/retention chemistry. Unless specified otherwise, it is intended that all stereoisomeric forms of the compounds of the present invention as well as enantiomerically enriched mixtures thereof, form part of the present invention.

Diastereomeric mixtures can be separated into their individual diastereomers on the basis of their physical chemical differences by methods well known to those skilled in the art, such as chromatography and/or fractional crystallization. A more detailed description of techniques that can be used to resolve stereoisomers of compounds from their racemic mixture can be found in Jean Jacques Andre Collet, Samuel H. Wilen, Enantiomers, Racemates and Resolutions, John Wiley and Sons, Inc. (1981).

Compounds of this invention can exist as one or more stereoisomers. The various stereoisomers include enantiomers and diastereomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. The compounds of the invention may be present as an enantiomerically enriched mixture of stereoisomers, individual stereo isomers or as an optically active form. For example, two possible enantiomers of Formula (1) are depicted as Formula 1a (the “S” enantiomer) and Formula 1 b (the “R” enantiomer) involving the isoxazoline chiral center identified with an asterisk (*). Molecular depictions drawn herein follow standard conventions for depicting stereochemistry.

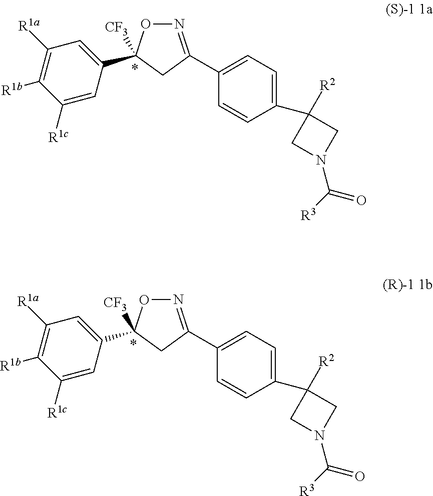

For illustrative purposes, the reaction schemes depicted below demonstrate potential routes for synthesizing key intermediates and compounds of the present invention. Those skilled in the art will appreciate that other suitable starting materials, reagents, and synthetic routes may be used to synthesize the intermediates and compounds of the present invention and a variety of derivatives thereof. Further, many of the compounds prepared by the methods described below can be further modified in light of this disclosure using conventional chemistry. Schemes 1-5 outline the general procedures useful for the preparation and isolation of compounds of the present invention. It is to be understood, however, that the invention, as fully described herein and as recited in the claims, is not intended to be limited by the details of the following schemes or modes of preparation.

In the preparation of compounds of the present invention, protection of remote functionality of intermediates from undesired reactions can be accomplished with a protecting group. The term “protecting group” or “PG” refers to a substituent that is commonly employed to block or protect a particular functionality while reacting other functional groups on the compound. For example, an amine-protecting group is a substituent attached to an amine that blocks or protects the amine-functionality of the compound or intermediate. Suitable amine protecting groups include: 1-tert-butyloxycarbonyl (Boc), acyl groups including: formyl, acetyl, chloroacetyl, trichloro-acetyl, o-nitrophenylacetyl, o-nitrophenoxyacetyl, trifluoroacetyl, acetoacetyl, 4-chlorobutyryl, isobutyryl, o-nitrocinnamoyl, picolinoyl, acylisothiocyanate, aminocaproyl, benzoyl, and the like; and acyloxy groups including: methoxycarbonyl, 9-fluorenyl-methoxycarbonyl, 2,2,2-trifluoroethoxycarbonyl, 2-trimethylsilylethxoycarbonyl, vinyloxycarbonyl, allyloxycarbonyl, 1,1-dimethyl-propynyloxycarbonyl, benzyloxy-carbonyl, p-nitrobenzyloxycarbony, 2,4-dichlorobenzyloxycarbonyl, and the like. Similarly, diphenylmethane and benzylcarbamates can be used as amine protecting groups. Suitable protecting groups and their respective uses are readily determined by one skilled in the art. For a general description of protecting groups and their use, see T. W. Greene, Protective Groups in Organic Synthesis, John Wiley & Sons, New York, 1991.

In the Schemes below, the following catalysts/reactants and miscellaneous abbreviations include: mobile phase (MP); supercritical fluid chromatography (SFC); N,N-dimethyl formamide (DMF); dimethyl acetamide (DMA); acetonitrile (ACN or Acn); formic acid (FA); dichloromethane (DCM); N-chloro-succinimide (NCS); ethanol (EtOH); methyl tert-butyl ether (MTBE); triethylamine (TEA); methanol (MeOH), tetrahydrofuran (THF); ethyl acetate (EtOAc); trifluoroacetic acid (TFA); triphenylphosphine palladium (Pd(PPh₃)₄); (2,2,6,6-tetramethylpiperidin-1-yl)oxyl (TEMPO); and diisobutylaluminium hydride (DIBAL-H); 4-dimethylaminopyridine (DMAP); potassium bis(trimethylsilyl) (KHMDS); N-chlorosuccinimide (NCS); 1,3-bis(diphenylphosphino)propane (DPPP); dimethyl sulfoxide (DMSO); amidecarbonyldiimidazole (CDl); (Bis-(2-methoxyethyl)aminosulfur trifluoride) (BAST); 1-hydroxybenzotriazole hydrate (HOBt); and N,N,N′,N′-Tetramethyl-O-(7-azabenzotriazol-1-yl)uronium hexafluorophosphate (HATU), mesyl chloride (MsCl); isopropylmagnesium chloride (iPrMgCl); t-butyloxycarbonyl (Boc); palladium(II) acetate (Pd(OAc)₂); lithium borohydride (LiBH₄); and aqueous (Aq).

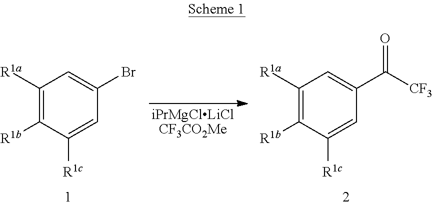

The requisite trifluophenylethanone derivatives 2 can be prepared according to Scheme 1. Metallation of the aryl bromide 1 under modified

Grignard conditions and addition to ethyl 2,2,2-trifluoroacetate provides the substituted trifluorophenylethanones 2.

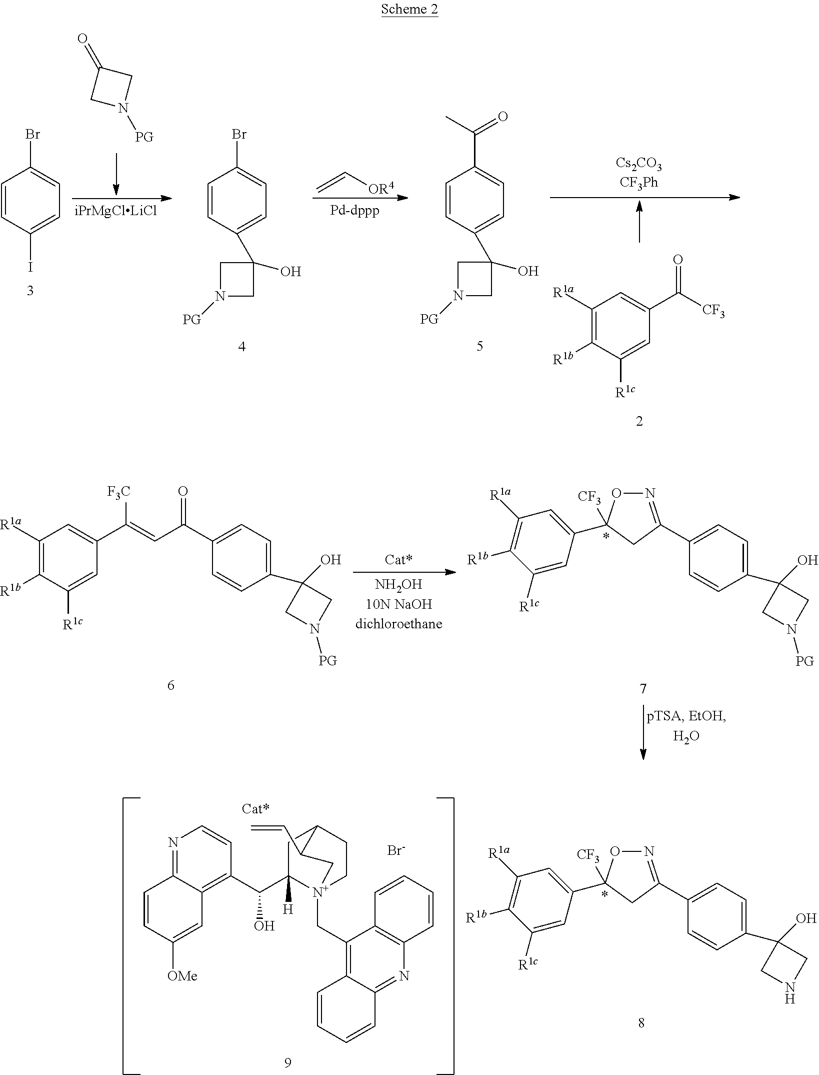

R^{1a}, R^{1b} and R^{1c} are as defined herein. The R⁴ substituent depicts a C₁-C₆alkyl moiety (e.g., methyl, ethyl, propyl, isopropyl, butyl, isobutyl and the like). PG is an amine protecting group, for example Boc, diphenylmethane, or a benzylcarbamate. The asterisk (*) depicts a chiral center (i.e., R or S stereochemistry).

A chiral synthesis of the compounds described within can be achieved according to Scheme 2. From iodobromobenzene 3, metallation and condensation with a protected azetidinone provides the hydroxyphenyl azetidine 4 in a one-pot reaction. Palladium catalyzed condensation with a vinyl ether provides the acetophenone 5 which can undergo condensation with a subsutituted trifluorophenylethanone derivative (2) to give the chalcone 6. Addition of hydroxylamine and cyclization in the presence of a quine-based chiral catalyst such as 9 provides the desired enantiomer of the isoxazoline 7. Removal of the nitrogen protecting group provides the chiral azetidine 8.

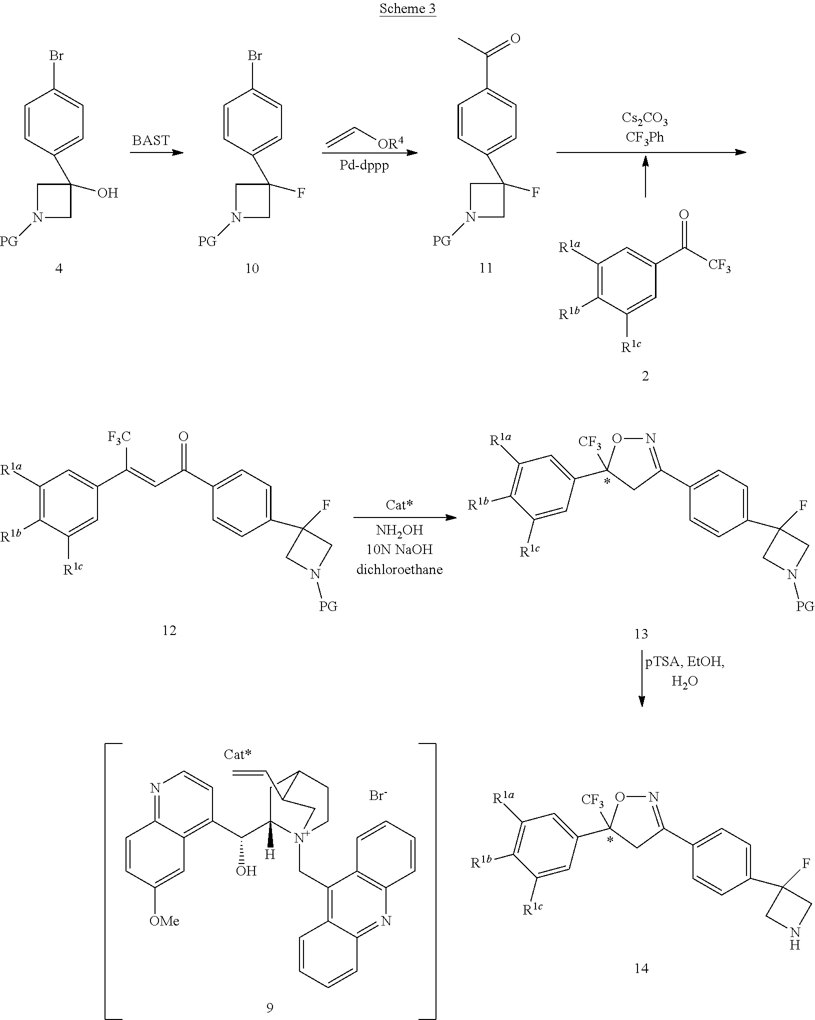

R^{1a}, R^{1b} and R^{1c} are as defined herein. The R⁴ substituent depicts a C₁-C₆alkyl moiety (e.g., methyl, ethyl, propyl, isopropyl, butyl, isobutyl and the like). PG is an amine protecting group, for example Boc, diphenylmethane, or a benzylcarbamate. The asterisk (*) depicts a chiral center, (i.e., R or S stereochemistry).

Alternatively, the hydroxyazetidine 4 can be treated with a fluorinating agent such as BAST, DAST or Xtalfluor to provide the fluoroazetidine 10. This can be carried on through the chiral synthesis as described in Scheme 2 to provide the chiral fluoroazetidinones 14.

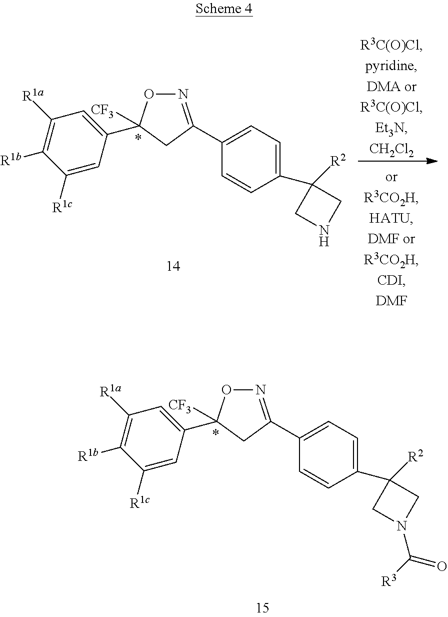

R^{1a}, R^{1b}, R^{1c},R² and R³ are as defined herein.

Amide analogs of the azetidine ring can be prepared as shown in Scheme 4. Acylation of the azetidine ring can be accomplished by reaction of the azetidine 14 with an acid chloride in pyridine/DMA or by a condensation with a carboxylic acid utilizing a condensing agent such as CDl, HATU or HOBt to afford the substituted azetidine 15.

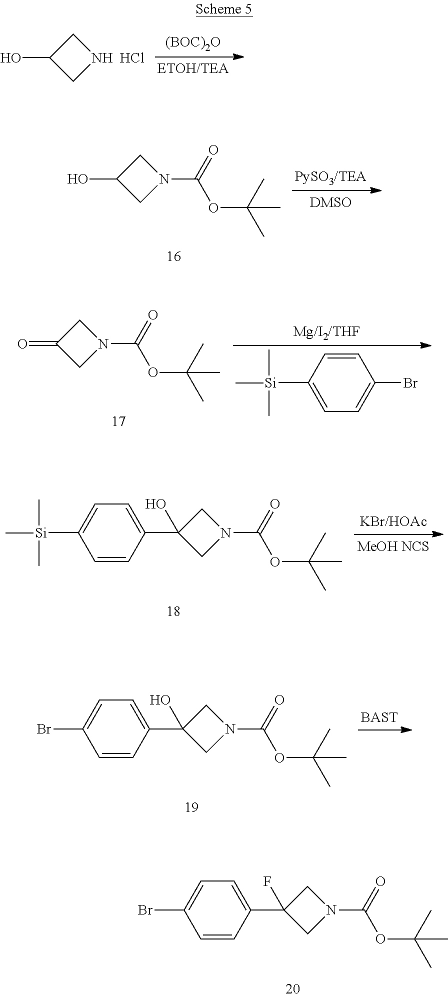

The phenyl azetidines can also be prepared as shown in Scheme 5. Boc protection of hydroxyazetidine hydrochloride followed by oxidation of the hydroxyl group gave the ketoazetidine 16. This could be condensed with bromoaryl silanes by formation of the aryl Grignard reagent and subsequent condensation with the ketone to provide the silyl phenyl azetadine 18.

Replacement of the silane with bromine was accomplished by treatment with potassium bromide in acetic acid to give the desired bromophenyl azetidine 19. Fluorination of the hydroxyazetidine 4 can be accomplished by treatment with BAST to provide 20.

The skilled person will appreciate that the compounds of the present invention could be made by methods other than those herein described as incorporated herein by reference, by adaptation of the methods herein described and/or adaptation of methods known in the art, for example the art described herein, or using standard textbooks such as “Comprehensive Organic Transformations—A Guide to Functional Group Transformations”, R C Larock, Wiley-VCH (1999 or later editions).

The compounds of the present invention (including the compositions and processes used therein) may also be used in the manufacture of a medicament for the therapeutic applications described herein.

The compounds of prepared by processes of the present invention, stereoisomers thereof, and compositions comprising a therapeutically effective amount of a Formula I compound and a veterinarily acceptable excipient, diluent, or carrier are useful as ectoparasiticides for the control and treatment of infections or infestations manifested by said ectoparasite in an animal. The compounds prepared by the processes described herein have utility as an ectoparasiticide, in particular, as an acaricide and insecticide. They may, in particular, be used in the fields of veterinary medicine, livestock husbandry and the maintenance of public health: against acarids, insects, and copepods which are parasitic upon vertebrates, particularly warm-blooded vertebrates, including companion animals, livestock, and fowl and cold-blooded vertebrates like fish.

Some non-limiting examples of acaride, insect, and copepod parasites include: ticks (e.g., Ixodes spp., Rhipicephalus spp., Boophilus spp., Amblyomma spp., Hyalomma spp., Haemaphysalis spp., Dermacentor spp., Ornithodorus spp., and the like); mites (e.g., Dermanyssus spp., Sarcoptes spp., Psoroptes spp., Chorioptes spp., Demodex spp., and the like); chewing and sucking lice (e.g., Damalinia spp., Linognathus spp., and the like); copepods (e.g., sea lice within the Order Siphonostomatoida, including genera Lepeophtheirus and Caligus); fleas (e.g., Siphonaptera spp., Ctenocephalides spp., and the like); biting flies and midges (e.g., Tabanidae spp., Haematobia spp., Stomoxys spp., Dermatobia spp., Simuliidae spp., Ceratopogonidae spp., Psychodidae spp., and the like); and bed bugs (e.g., insects within the genus Cimex and family Cimicidae).

The compounds of the invention can also be used for the treatment of endoparasites, for example, heartworms, roundworms, hookworms, whipworms, and tapeworms.

The compounds prepared according to processes described herein and compositions comprising said compounds in conjunction with at least one other veterinary agent are of particular value in the control of ectoparasites, endoparasites, and insects which are injurious to, or spread or act as vectors of diseases in companion animals, livestock, and fowl. The ectoparasites, insects, and endoparasites which can be treated with a combination of a Formula (1) compound and an additional veterinary agent include those as herein before described and including helminthes of the phylum platyhelminthes (e.g., trematodes, eucestoda, and cestoda), and nemathelminthes (e.g., nematodes).

Any of the compounds of the present invention, or a suitable combination of a compound of the present invention and optionally, with at least one additional veterinary agent may be administered directly to the animal and/or indirectly by applying it to the local environment in which the animal dwells (such as bedding, enclosures, and the like). Direct administration includes contacting the skin, fur, or feathers of a subject animal with the compound(s), or by feeding or injecting the compounds into the animal.

The Formula (1) compounds, stereoisomers thereof, and combinations with at least one additional veterinary agent, as described herein, are of value for the treatment and control of the various lifecycle stages of insects and parasites including egg, nymph, larvae, juvenile and adult stages.

The present invention also relates to a method of administering a compound of the present invention alone or in combination with at least one additional veterinary agent, and optionally a veterinarily acceptable excipient, diluent, or carrier, to animals in good health comprising the application to said animal to reduce or eliminate the potential for human parasitic infection or infestation from parasities carried by the animal and to improve the environment in which the animals inhabit.

### EXAMPLES The following intermediates and Examples were prepared according to the Schemes as described above. Further, additional synthesis information for the intermediates is described in WO2012/017359.

Preparation of the Silyl Phenyl Azetidines

Preparation 1a: tert-butyl-3-hydroxyazetidine-1-carboxylate

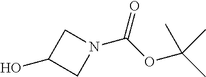

To a stirred cold (0° C.) solution of 3-hydroxyazetidine hydrochloride (75 g, 0.68 mol) in ethanol (1300 mL) was added triethylamine (208 g/280 mL, 2.05 mol) followed by Boc₂O (164 g, 0.75 mol). The resultant solution was stirred at ambient temperature for 16 hours. GC/MS analysis of the reaction mixture revealed complete reaction. Volatiles were removed in vacuo and the residue was diluted with EtOAc (1300 mL) and washed with 10% citric acid (700 mL), water (700 mL) and brine (700 mL). The organics were dried over sodium sulfate filtered, and concentrated to give the desired product (100.8 g, 85% yield).¹H NMR (CDCl₃) δ 4.6; (m, 1 H), 4.2; (m, 2 H), 3.8; (m, 2 H), 1.4; (s, 9 H).

Preparation 2a: tert-butyl 3-oxoazetidine-1-carboxylate

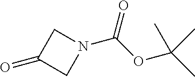

A 5L-3-neck flask equipped with mechanical stirrer, thermocouple, addition funnel and nitrogen inlet was charged with Py-SO₃ (277 g, 1.74 mol) and DMSO (900 mL) and cooled to 10° C. in ice-bath. TEA (177 g/244 mL, 1.74 mol) was added. A solution of tert-butyl-3 hydroxyazetidine-1-carboxylate (Preparation 1a, 100.8 g, 0.58 mol) in DMSO (500 mL) was added slowly via addition funnel at 10° C. The reaction was stirred at ambient temperature overnight. GC/MS analysis of the reaction mixture reveals that the reaction was completed. The reaction was quenched with brine (1 L). Solids were filtered and the aqueous was extracted with ethyl acetate (3×1 L). The combined organics were washed with saturated aqueous NaHCO3 (1.5 L), brine (1.5 L), dried over sodium sulfate, filtered, and concentrated to give the desired product (94 g, 95% yield). ¹H NMR (CDCl₃) δ 4.6; (s, 4 H), 1.4; (s, 9 H).

Preparation 3a: tert-butyl 3-hydroxy-3-(4-(trimethylsilyl)phenyl)azetidine-1-carboxylate

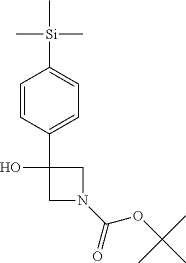 [SMILES: CC(C)(C)OC(=O)N1CC(O)(c2ccc([Si](C)(C)C)cc2)C1]

A 2L-3neck flask equipped with mechanical stirrer, thermocouple, addition funnel and nitrogen inlet was charged with (4-bromophenyl)trimethylsilane (80.4 g, 0.35 mol), THF (600 mL), Mg (8.5 g), and I₂ (catalytic amount). The suspension was refluxed at 68° C. for 1.5 hours until all magnesium disappeared. The solution was cooled to 0° C. in an ice-bath. Then, a solution of tert-butyl 3-oxoazetidine-1-carboxylate (Preparation 2a, 30 g, 0.17 mol) in THF (200 mL) was added slowly via addition funnel. The solution was stirred at 0° C. for 3 hours. LC/MS indicated the formation of desired product. The reaction was quenched with brine at 0° C. The aqueous layer was extracted with EtOAc (2×800 mL). The combined organics were dried over sodium sulfate, filtered and concentrated to give the desired product (47.4 g, 84% yield). ¹H NMR (CDCl₃) δ 7.3; (d, 2 H), 7.2; (d, 2 H), 4.0; (d, 2 H), 3.9; (d, 2 H), 2.9; (s, 1 H), 1.2; (s, 9 H), 0.0; (s, 9 H).

Preparation 4a: tert-butyl 3-(4-bromophenyl)-3-hydroxyazetidine-1-carboxylate

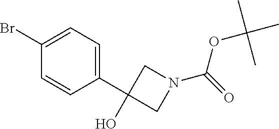 [SMILES: CC(C)(C)OC(=O)N1CC(O)(c2ccc(Br)cc2)C1]

20

A mixture of tert-butyl 3-hydroxy-3-(4-(trimethylsilyl)phenyl)azetidine-1-carboxylate (Preparation 3a, 45 g, 0.14 mol) and KBr (25 g, 0.21 mol) in acetic acid (1 L) and MeOH (100 mL) was heated at 60° C. for 20 minutes. Then N-chlorosuccinimide (22.4 g, 0.17 mol) was added to the reaction mixture and stirred at 60° C. for 2 hours. LC/MS indicated the reaction was complete (only product peak). After cooling to ambient temperature, the mixture was poured into ice-water (1 L). The mixture was extracted with CHCl₃ (2×800 mL). The combined organics were washed with 3M NaOH (2×600 mL), water (600 mL), dried over sodium sulfate, filtered and concentrated. The crude product was washed with ether to afford the desired product (35 g, 76% yield). ¹H NMR (CDCl₃) δ 7.5; (d, 2 H), 7.4; (d, 2 H), 4.2; (s, 4 H), 3.4; (s, 1 H), 1.4; (s, 9 H).

Preparation 5a: tert-butyl-3-(4-bromophenyl)-3-fluoroazetidine-1-carboxylate

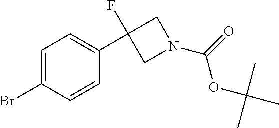 [SMILES: CC(C)(C)OC(=O)N1CC(F)(c2ccc(Br)cc2)C1]

Tert-butyl-3-(4-bromophenyl)-3-hydroxyazetidine-1-carboxylate (Preparation 4a, 25 g, 0.076 mol) in CH₂Cl₂ (500 mL) was cooled to −78° C. To this slurry was slowly added BAST (20.2 g, 0.09 mol) via addition funnel. The temperature of the reaction was increased slowly from −78° C. to ambient temperature. The mixture was stirred at ambient temperature overnight. LC/MS indicated that the reaction was complete. The reaction was quenched with saturated aqueous NaHCO₃ solution (500 mL) and 1M NaOH (500 mL). The aqueous layer was extracted with CH₂Cl₂ (2×800 mL). The combined organics were washed with aqueous citric acid (2×700 mL), dried over Na₂SO₄, filtered, and concentrated to afford the desired product as tan solid (24.4 g, 97% yield). ¹H NMR (CDCl₃) δ 7.5; (d, 2 H), 7.3; (d, 2 H), 4.4; (m, 2 H), 4.2; (m, 2 H), 1.4; (s, 9 H).

Preparation of 3-(4-acetyl-phenyl)-3-fluoro-azetidine-1-carboxylic acid tert-butyl ester

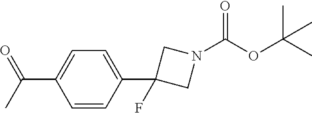 [SMILES: CC(=O)c1ccc(C2(F)CN(C(=O)OC(C)(C)C)C2)cc1]

In 100 mL autoclave vessel a solution of tert-butyl-3-(4-bromophenyl)-3-fluoroazetidine-1-carboxylate (5g, 15.142 mmol, 1 eq) in ethanol (17.5 mL) was degassed with nitrogen gas for 30 minutes at room temperature. triethylamine (3.79 mL, 27.256 mmol, 1.8 eq), butyl vinyl ether (BVE, 3.91 mL, 30.282 mmol, 2 eq), 1,3-bis(diphenylphosphino)propane (DPPP, 0.375 g, 0.909 mmol, 0.06 eq) were added followed by addition of Pd(OAc)₂ (0.102 g, 0.454 mmol, 0.03 eq.) at room temperature. Resulting reaction mixture was heated at 96° C. for 16 hours in an autoclave. After complete consumption of starting material, the reaction mixture was quenched with 1N HCl (5 mL, pH˜2-3) and stirred for 2 hours at room temperature. After 2 hours, pH of reaction mixture was adjusted to 7 by addition of saturated NaHCO₃ solution and extracted with ethyl acetate (3×50 mL). Combined organic layer was washed with brine (250 mL), dried over sodium sulphate and concentrated under reduced pressure to get crude compound as dark brown sticky oil (6.1 g, crude). Crude compound was purified by column chromatography on silica gel using 230-400 silica mesh. Desired compound was eluted in 10% ethyl acetate in n-hexane to give product as off white semi-solid (2.56 g, 57.66%). ¹H NMR (400 MHz, CDCl3)δ: 1.46 (s, 9H), 2.61; (s, 3H), 4.20; (dd, J₁=0.88 Hz, J₂=10.28 Hz, 1H), 4.24; (dd, J₁=0.92 Hz, J₂=10.36 Hz, 1H), 4.39; (dd, J₁=1.28 Hz, J₂=10.24 Hz, 1H), 4.44; (dd, J₁=1.28 Hz, J₂=10.20 Hz, 1H), 7.55; (dd, J₁=1.48 Hz, J₂=8.48 Hz, 2H), 8.00; (d, J=7.96 Hz, 2H), LC-MS (m/z): =294.1 (M+H).

Chalcones

Preparation of (Z)-tert-butyl 3-(4-(3-(3,5-dichloro-phenyl)-4,4,4-trifluoro-but-2-enoyl)phenyl)-3-fluoroazetidine-1-carboxylate (C-1)

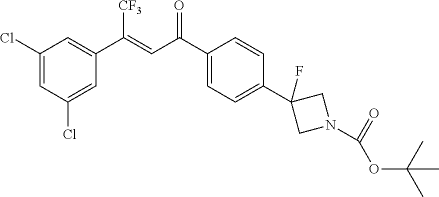 [SMILES: CC(C)(C)OC(=O)N1CC(F)(c2ccc(C(=O)/C=C(/c3cc(Cl)cc(Cl)c3)C(F)(F)F)cc2)C1]

In 25 mL two neck RBF equipped with Dean-stark apparatus, to the stirred solution of 1-(3,5-dichloro-phenyl)-2,2,2-trifluoro-ethanone (2.56 g, 8.727 mmol, 1 eq.) in toluene (18 mL) and 1,1,1-trifluoromethyl benzene (18 mL) was added 3-(4-acetyl-phenyl)-3-fluoro-azetidine-1-carboxylic acid tert-butyl ester (2.43 g,10.036 mmol, 1.15 eq) and Cs₂CO₃ (0.284 g,0.873 mmol, 0.1 eq.) at room temperature. Resulting reaction mixture was stirred at 110° C. for 16 hours. After complete consumption of starting material, reaction mixture cooled to room temperature and was diluted with tert-butylmethyl ether (30 mL) and filtered through bed of celite. Filtrate was concentrated in vacuo to get crude compound as brown sticky oil (4.12 g, crude). Crude compound was purified by column chromatography on silica gel using 230-400 mesh. Desired compound was eluted in 20% ethyl acetate in n-hexane to give product as light yellow solid (2.2 g, 48.67%).¹H NMR (400 MHz, CDCl3)δ: 1.47; (s, 9H), 4.09-4.14; (m, 1H), 4.17-4.22; (m, 1H), 4.37-4.40; (m, 1H), 4.42-4.45; (m, 1H), 7.13; (d, J=1.68 Hz, 2H), 7.31; (t, J=1.84 Hz, 1 H), 7.38; (d, J=1.32 Hz, 1 H), 7.56; (d, J=8.4 Hz, 2H), 7.86; (d, J=8.36 Hz, 2H). LC-MS (m/z): =516.0 (M−H).

Preparation of (Z)-tert-butyl 3-(4-(3-(3,5-dichloro-4-fluoro-phenyl)-4,4,4-trifluoro-but-2-enoyl)-phenyl)-3-fluoro-azetidine-1-carboxylate (C-2)

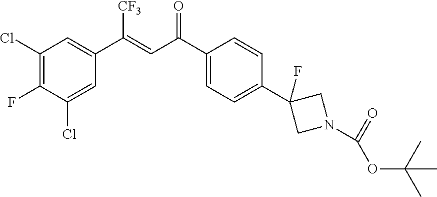 [SMILES: CC(C)(C)OC(=O)N1CC(F)(c2ccc(C(=O)/C=C(/c3cc(Cl)c(F)c(Cl)c3)C(F)(F)F)cc2)C1]

This compound was prepared by using procedure similar to that of C-1 except that 1-(3,5-dichloro-4-fluoro-phenyl)-2,2,2-trifluoro-ethanone was used in place of 1-(3,5-dichloro-phenyl)-2,2,2-trifluoro-ethanone to yield 4.1 g (64.06%). ¹H NMR (400 MHz, CDCl3)δ: 1.47; (s, 9H), 4.16; (d, J=10.32 Hz, 1H), 4.21; (d, J=10.44 Hz, 1H), 4.39; (d, J=10.52 Hz, 1H), 4.45; (d, J=10.32 Hz, 1H), 7.23; (d, J=6.08 Hz, 2H), 7.40; (d, J=1.08 Hz, 1 H), 7.58; (d, J=8.4Hz, 2H), 7.88; (d, J=8.28 Hz, 2H). LC-MS (m/z): =535.9 (M+H).

Preparation of (Z)-tert-butyl 3-(4-(3-(3,4,5-trichloro-phenyl)-4,4,4-trifluoro-but-2-enoyl)-phenyl)-3-fluoro-azetidine-1-carboxylate (C-3)

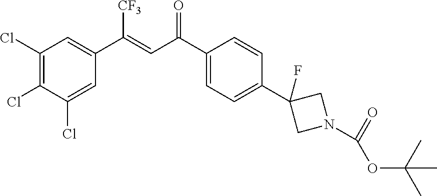 [SMILES: CC(C)(C)OC(=O)N1CC(F)(c2ccc(C(=O)/C=C(/c3cc(Cl)c(Cl)c(Cl)c3)C(F)(F)F)cc2)C1]

This compound was prepared by using procedure similar to that of C-1 except that 2,2,2-trifluoro-1-(3,4,5-trichloro-phenyl)-ethanone was used in place of 1-(3,5-dichloro-phenyl)-2,2,2-trifluoro-ethanone to yield 4.5g (68.18%). ¹H NMR (400 MHz, CDCl3)δ: 1.47 (s, 9H), 4.16; (d, J=10.4 Hz, 1 H), 4.21; (d, J=10.32 Hz, 1 H), 4.39; (d, J=10.68 Hz, 1 H), 4.44; (d, J=9.92 Hz, 1 H), 7.28; (s, 2H), 7.42; (d, J=1.12 Hz, 1H), 7.56-7.58; (m, 2H), 7.87; (d, J=8.24Hz, 2H). LC-MS (m/z): =550.1 (M-H).

### Example 1. (R)-tert-butyl 3-(4-(5-(3,5-dichloro-4-fluorophenyl)-5-(trifluoromethyl)-4,5-dihydroisoxazol-3-yl)phenyl)-3-fluoroazetidine-1-carboxylate

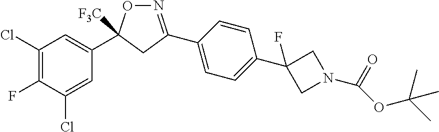 [SMILES: CC(C)(C)OC(=O)N1CC(F)(c2ccc(C3=NO[C@](c4cc(Cl)c(F)c(Cl)c4)(C(F)(F)F)C3)cc2)C1]

The chalcone, C-2, (200 mg, 0.37 mmol) and catalyst 9 (1 mg, 0.02 mmol) were dissolved dichloroethane (1.2 mL) and cooled to 0° C. In a separate flask, 10N solution of aqueous sodium hydroxide was cooled to 0° C. and a solution of hydroxylamine hydrochloride (52 mg, 0.75 mmol) in water (0.2 mL) was added while maintaining the temperature at less than 5° C. The base solution was added to the dichloroethane solution and the resulting biphasic mixture was stirred at 0° C. for 90 minutes. The aqueous phase was discarded. The organics were filtered through a plug of silica gel (2 gm) which was eluted with 10% methyl-tert-butyl ether/dichloroethane. The material was concentrated under vacuum to 180mg of a white solid. MS M-H=550. Chiral LC shows an 85:15 ratio of S and R isomers[Chiralcel AD-3R, 150×4.6 mm, 3 micron column, 1.5 mL/min, 260 nm detection, 40° C., ramp of 5% acetonitrile to 100% in methanol over 10 minutes; elution times: S-isomer (1.96 minutes), R-isomer (3.04 minutes). ¹H NMR (600 MHz, CDCl3)δ: 1.51; (s, 9H), 3.72; (d, J=12 Hz, 1H), 4.12; (d, J=12 Hz, 1H), 4.23; (d, d J=12 Hz, 2H), 4.45; (d,d J=12 Hz, 2H), 7.57; (d, J=8 Hz, 2H), 7.62; (d, J=8 Hz, 2H), 7.75; (d, J=8 Hz, 2H).

We claim:

1. A process for the preparation of a compound having the Formula (1) 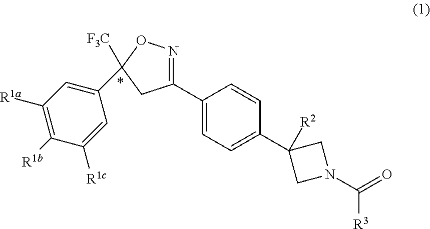
wherein
R^{1a}, R^{1b}, and R^{1c} are each independently hydrogen, halo, or C₁-C₆haloalkyl;
R² is hydroxyl, or fluoro;
R³ C₁-C₆alkyl, C₂-C₆alkenyl, C₀-C₆alkylC₃-C₆cycloalkyl, C₀-C₆alkylphenyl, C₀-C₆alkylheteroaryl, or C₀-C₆alkylheterocycle;
wherein R³ C₁-C₆alkyl or C₀-C₆alkylC₃-C₆cycloalkyl moiety can be optionally and independently substituted by at least one substituent selected from cyano, halo, hydroxyl, oxo, C₁-C₆alkoxy, C₁-C₆haloalkoxy, C₁-C₆haloalkyl, —S(O)ₙR^{c}, —SH, —S(O)ₙNR^{a}R^{b}, —NR^{a}R^{b}, —NR^{a}C(O)R^{b}, —SC(O)R, —SCN, or —C(O)NR^{a}R^{b}, and the C₀-C₆alkylC₃-C₆cycloalkyl moiety can be further substituted with C₁-C₆alkyl or hydroxyC₁-C₆alkyl-; and
wherein R³ C₀-C₆alkylphenyl, C₀-C₆alkylheteroaryl, or C₀-C₆alkylheterocycle moiety can be further optionally substituted with at least one substituent selected from cyano, halo, oxo, ═S, hydroxyl, C₁-C₆alkoxy, C₁-C₆alkyl, C₁-C₆haloalkyl, —SH, —S(O)ₙR, and C₁-C₆haloalkoxy;
R is C₁-C₆alkyl or C₃-C₆cycloalkyl optionally substituted with at least one halo substituent;
R^{a} is hydrogen, C₁-C₆alkyl, or C₀-C₃alkylC₃-C₆cycloalkyl; wherein the alkyl and alkylcycloalkyl is optionally substituted by cyano or at least one halo substituent;
R^{b} is hydrogen, C₁-C₆alkyl, C₃-C₆cycloalkyl, C₀-C₃alkylphenyl, C₀-C₃alkylheteroaryl, or C₀-C₃alkylheterocycle, each optionally substituted, where chemically possible, with at least one substituent selected from hydroxyl, cyano, halo, or —S(O)ₙR;
R^{c} is C₁-C₆alkyl, C₁-C₆haloalkyl, C₁-C₆haloalkylC₃-C₆cycloalkyl, C₀-C₃alkylC₃-C₆cycloalkyl, C₀-C₃alkylphenyl, C₀-C₃alkylheteroaryl, or C₀-C₃alkylheterocycle each optionally substituted with at least one substituent selected from cyano, halo, hydroxyl, oxo, C₁-C₆alkoxy, C₁-C₆haloalkoxy, C₁-C₆haloalkyl, —S(O)ₙR, —SH, —S(O)ₙNR^{a}R^{b}, —NR^{a}R^{b}, —NR^{a}C(O)R^{b}, —SC(O)R, —SCN, or —C(O)NR^{a}R^{b};
n is the integer 0, 1, or 2; and
* depicts a chiral center;
stereoisomers thereof, and veterinarily acceptable salts thereof;
said process comprising, optionally in a solvent:
a) metallating 1-bromo-4-iodobenzene with a Grignard reagent or halogen-metal exchange with an alkyllithium and reacting metallated species with a protected azetidinone in a one-pot process or in a step-wise process to provide a protected 3-(4-bromophenyl)azetidin-3-ol, wherein PG is an amine protecting group; 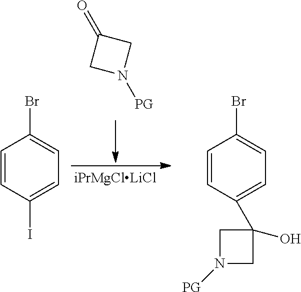
b) optionally fluorinating the resulting protected hydroxyazetidine by treatment with a fluorinating agent to provide a protected fluoroazetidine; 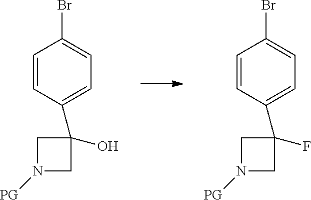
c) palladium catalyzed condensation of the bromophenylazetidine from steps a or b, above, with a vinyl ether to provide a protected 1-(4-(azetidin-3-yl)phenyl)ethanone derivative, wherein R² is hydroxyl or fluoro and R⁴ is a C₁-C₆alkyl; 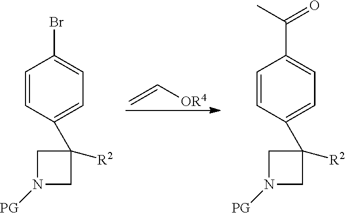
d) condensation of the 1-(4-(azetidin-3-yl)phenyl)ethanone derivative with a substituted trifluorophenylethanone to provide a 1-(4-(azetidin-3-yl)phenyl)-4,4,4-trifluoro-3-phenylbut-2-en-1-one analog; 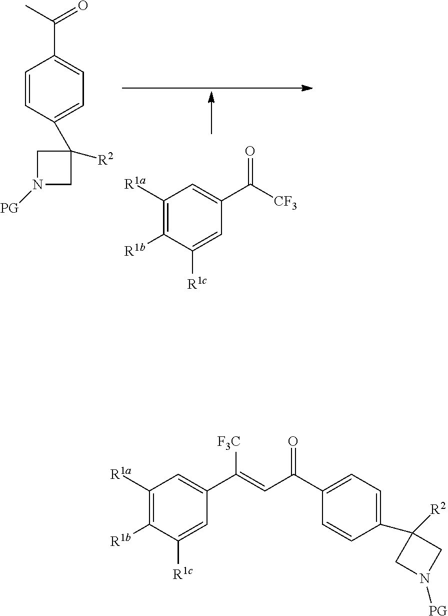
e) addition of hydroxylamine to the 1-(4-(azetidin-3-yl)phenyl)-4,4,4-trifluoro-3-phenylbut-2-en-1-one analog and cyclization in the presence of a quinine based chiral catalyst to provide a protected 3-(4-(azetidin-3-yl)phenyl)-5-phenyl-5-(trifluoromethyl)-4,5-dihydroisoxazole analog, wherein * denotes a chiral center; 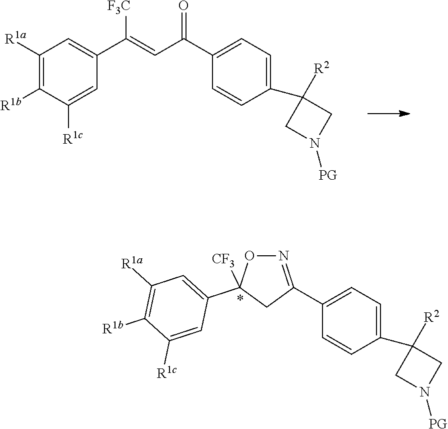
f) removal of the azetidine protecting group to provide a 3-(4-(azetidin-3-yl)phenyl)-5-phenyl-5-(trifluoromethyl)-4,5-dihydroisoxazole analog; and 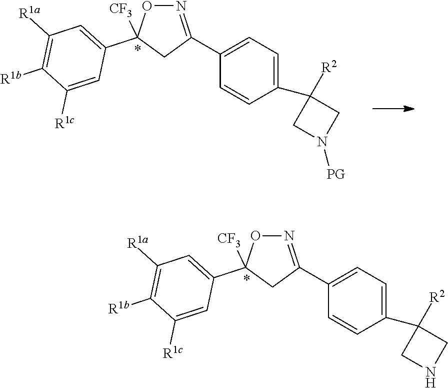
g) Coupling the 3-(4-(azetidin-3-yl)phenyl)-5-phenyl-5-(trifluoromethyl)-4,5-dihydroisoxazole analog with an acid or acid chloride under standard amide formation conditions.

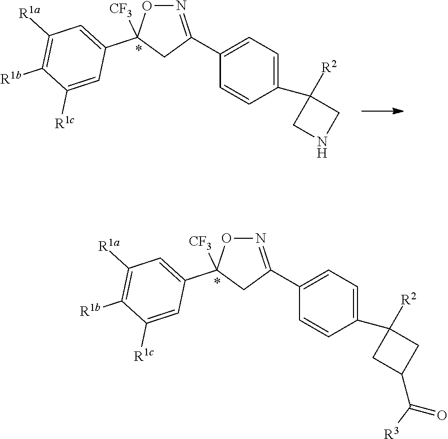

2. The process of claim 1 wherein
R² is fluoro;
stereoisomers thereof, and veterinarily acceptable salts thereof.

3. The process of claim 2 wherein
R^{1a}, R^{1b}, and R^{1c} are each independently hydrogen, chloro, fluoro, bromo, or trifluoromethyl;
R³ is methyl, ethyl, propyl, isopropyl, butyl, isobutyl, t-butyl, cyclopropyl, or cyclobutyl, wherein each substituent can be optionally and independently substituted by at least one substituent selected from halo, hydroxyl, C₁-C₆haloalkyl, or —S(O)ₙR^{c}; and wherein the cyclopropyl and cyclobutyl can further be optionally substituted with C₁-C₆alkyl or hydroxyC₁-C₆alkyl-;
or R³ is thietanyl, thietanyl-1-oxide, thietanyl-1,1-dioxide, pyrazolyl, —CH₂-pyridyl or —CH₂pyrazolyl, wherein each substituent can be further optionally substituted with at least one substituent selected from halo, or C₁-C₆alkyl; and
R^{c} is C₁-C₄ alkyl;
stereoisomers thereof, and veterinarily acceptable salts thereof.

4. The process of claim 3 wherein
R^{1a} and R^{1c} are each chloro, R^{1b} is fluoro, chloro or hydrogen;
R³ is —CH₂S(O)₂CH₃ or thietan-3-yl-1,1-dioxide;
stereoisomers thereof, and veterinarily acceptable salts thereof

5. The process of claim 1 wherein
R² is hydroxy;
stereoisomers thereof, and veterinarily acceptable salts thereof.

6. The process of claim 5 wherein
R^{1a}, R^{1b}, and R^{1c} are each independently hydrogen, chloro, fluoro, bromo, or trifluoromethyl;
R³ is methyl, ethyl, propyl, isopropyl, butyl, isobutyl, t-butyl, cyclopropyl, or cyclobutyl, wherein each substituent can be optionally and independently substituted by at least one substituent selected from halo, hydroxyl, C₁-C₆haloalkyl, or —S(O)ₙR^{c}; and wherein the cyclopropyl and cyclobutyl can further be optionally substituted with C₁-C₆alkyl or hydroxyC₁-C₆alkyl-;
or R³ is thietanyl, thietanyl-1-oxide, thietanyl-1,1-dioxide, pyrazolyl, —CH₂— pyridyl or —CH₂pyrazolyl, wherein each substituent can be further optionally substituted with at least one substituent selected from halo, or C₁-C₆alkyl; and
R^{c} is C₁-C₄ alkyl;
stereoisomers thereof, and veterinarily acceptable salts thereof.

7. The process of claim 6 wherein
R^{1a} and R^{1c} are each chloro, R^{1b} is fluoro, chloro or hydrogen;
R³ is —CH₂S(O)₂CH₃ or thietan-3-yl-1,1-dioxide;
stereoisomers thereof, and veterinarily acceptable salts thereof.

8. The process of claim 1 wherein the chiral quinine-based catalyst is selected from:
(2S)-1-(acridin-9-ylmethyl)-2-((R)-hydroxy(6-methoxyquinolin-4-yl)methyl)-5-vinylquinuclidin-1-ium bromide;
(2S)-1-(acridin-9-ylmethyl)-2-((R)-hydroxy(6-methoxyquinolin-4-yl)methyl)-5-vinylquinuclidin-1-ium chloride;
(2S)-1-(anthracen-9-ylmethyl)-2-((R)-hydroxy(6-methoxyquinolin-4-yl)methyl)-5-vinylquinuclidin-1-ium bromide; and
(2S)-1-(anthracen-9-ylmethyl)-2-((R)-hydroxy(6-methoxyquinolin-4-yl)methyl)-5-vinylquinuclidin-1-ium chloride.

9. The process of claim 8 wherein the chiral quinine-based catalyst is selected from :
(2S)-1-(acridin-9-ylmethyl)-2-((R)-hydroxy(6-methoxyquinolin-4-yl)methyl)-5-vinylquinuclidin-1-ium bromide; or
(2S)-1-(acridin-9-ylmethyl)-2-((R)-hydroxy(6-methoxyquinolin-4-yl)methyl)-5-vinylquinuclidin-1-ium chloride.

10. The process of claim 9 wherein the chiral quinine-based catalyst is (2S)-1-(acridin-9-ylmethyl)-2-((R)-hydroxy(6-methoxyquinolin-4-yl)methyl)-5-vinylquinuclidin-1-ium bromide.

11. A compound selected from the group consisting of:
tert-butyl 3-(4-bromophenyl)-3-hydroxyazetidine-1-carboxylate;
benzyl 3-(4-bromophenyl)-3-hydroxyazetidine-1-carboxylate;
1-benzhydryl-3-(4-bromophenyl)azetidin-3-ol;
tert-butyl 3-(4-bromophenyl)-3-fluoroazetidine-1-carboxylate;
benzyl 3-(4-bromophenyl)-3-fluoroazetidine-1-carboxylate;
1-benzhydryl-3-(4-bromophenyl)-3-fluoroazetidine;
tert-butyl 3-(4-acetylphenyl)-3-hydroxyazetidine-1-carboxylate;
benzyl 3-(4-acetylphenyl)-3-hydroxyazetidine-1-carboxylate;
1-(4-(1-benzhydryl-3-hydroxyazetidin-3-yl)phenyl)ethanone;
tert-butyl 3-(4-acetylphenyl)-3-fluoroazetidine-1-carboxylate;
benzyl 3-(4-acetylphenyl)-3-fluoroazetidine-1-carboxylate;
1-(4-(1-benzhydryl-3-fluoroazetidin-3-yl)phenyl)ethanone;
(E/Z)-tert-butyl 3-(4-(3-(3,5-dichloro-4-fluorophenyl)-4,4,4-trifluorobut-2-enoyl)phenyl)-3-hydroxyazetidine-1-carboxylate;
(E/Z)-tert-butyl 3-hydroxy-3-(4-(4,4,4-trifluoro-3-(3,4,5-trichlorophenyl)but-2-enoyl)phenyl)azetidine-1-carboxylate;
(E/Z)-tert-butyl 3-(4-(3-(3,5-dichlorophenyl)-4,4,4-trifluorobut-2-enoyl)phenyl)-3-hydroxyazetidine-1-carboxylate;
(E/Z)-tert-butyl 3-(4-(3-(3,4-dichloro-5-(trifluoromethyl)phenyl)-4,4,4-trifluorobut-2-enoyl)phenyl)-3-hydroxyazetidine-1-carboxylate;
(E/Z)-benzyl 3-(4-(3-(3, 5-dichloro-4-fluorophenyl)-4,4,4-trifluorobut-2-enoyl)phenyl)-3-hydroxyazetidine-1-carboxylate;
(E/Z)-benzyl 3-hydroxy-3-(4-(4,4,4-trifluoro-3-(3,4,5-trichlorophenyl)but-2-enoyl)phenyl)azetidine-1-carboxylate;
(E/Z)-benzyl 3-(4-(3-(3,5-dichlorophenyl)-4,4,4-trifluorobut-2-enoyl)phenyl)-3-hydroxyazetidine-1-carboxylate;
(E/Z)-benzyl 3-(4-(3-(3,4-dichloro-5-(trifluoromethyl)phenyl)-4,4,4-trifluorobut-2-enoyl)phenyl)-3-hydroxyazetidine-1-carboxylate;
(E/Z)-1-(4-(1-benzhydryl-3-hydroxyazetidin-3-yl)phenyl)-3-(3,5-dichloro-4-fluorophenyl)-4,4,4-trifluorobut-2-en-1-one;
(E/Z)-1-(4-(1-benzhydryl-3-hydroxyazetidin-3-yl)phenyl)-3-(3,5-dichlorophenyl)-4,4,4-trifluorobut-2-en-1-one;
(E/Z)-1-(4-(1-benzhydryl-3-hydroxyazetidin-3-yl)phenyl)-4,4,4-trifluoro-3-(3,4,5-trichlorophenyl)but-2-en-1-one;
(E/Z)-1-(4-(1-benzhydryl-3-hydroxyazetidin-3-yl)phenyl)-3-(3,4-dichloro-5-(trifluoromethyl)phenyl)-4,4,4-trifluorobut-2-en-1-one;
(E/Z)-tert-butyl 3-(4-(3-(3,5-dichloro-4-fluorophenyl)-4,4,4-trifluorobut-2-enoyl)phenyl)-3-fluoroazetidine-1-carboxylate;
(E/Z)-tert-butyl 3-fluoro-3-(4-(4,4,4-trifluoro-3-(3,4,5-trichlorophenyl)but-2-enoyl)phenyl)azetidine-1-carboxylate;
(E/Z)-tert-butyl 3-(4-(3-(3,5-dichlorophenyl)-4,4,4-trifluorobut-2-enoyl)phenyl)-3-fluoroazetidine-1-carboxylate;
(E/Z)-tert-butyl 3-(4-(3-(3,4-dichloro-5-(trifluoromethyl)phenyl)-4,4,4-trifluorobut-2-enoyl)phenyl)-3-fluoroazetidine-1-carboxylate;
(E/Z)-benzyl 3-(4-(3-(3,5-dichloro-4-fluorophenyl)-4,4,4-trifluorobut-2-enoyl)phenyl)-3-fluoroazetidine-1-carboxylate;
(E/Z)-benzyl 3-fluoro-3-(4-(4,4,4-trifluoro-3-(3,4, 5-trich lorophenyl)but-2-enoyl)phenyl)azetidine-1-carboxylate;
(E/Z)-benzyl 3-(4-(3-(3,5-dichlorophenyl)-4,4,4-trifluorobut-2-enoyl)phenyl)-3-fluoroazetidine-1-carboxylate;
(E/Z)-benzyl 3-(4-(3-(3,4-dichloro-5-(trifluoromethyl)phenyl)-4,4,4-trifluorobut-2-enoyl)phenyl)-3-fluoroazetidine-1-carboxylate;
(E/Z)-1-(4-(1-benzhydryl-3-fluoroazetidin-3-yl)phenyl)-3-(3,5-dichloro-4-fluorophenyl)-4,4,4-trifluorobut-2-en-1-one;
(E/Z)-1-(4-(1-benzhydryl-3-fluoroazetidin-3-yl)phenyl)-3-(3,5-dichlorophenyl)-4,4,4-trifluorobut-2-en-1-one;
(E/Z)-1-(4-(1-benzhydryl-3-fluoroazetidin-3-yl)phenyl)-4,4,4-trifluoro-3-(3,4,5-trichlorophenyl)but-2-en-1-one;
(E/Z)-1-(4-(1-benzhydryl-3-fluoroazetidin-3-yl)phenyl)-3-(3,4-dichloro-5-(trifluoromethyl)phenyl)-4,4,4-trifluorobut-2-en-1-one;
(S)-tert-butyl 3-(4-(5-(3,5-dichloro-4-fluorophenyl)-5-(trifluoromethyl)-4,5-dihydroisoxazol-3-yl)phenyl)-3-hydroxyazetidine-1-carboxylate;
(S)-tert-butyl 3-(4-(5-(3,5-dichlorophenyl)-5-(trifluoromethyl)-4,5-dihydroisoxazol-3-yl)phenyl)-3-hydroxyazetidine-1-carboxylate;
(S)-tert-butyl 3-hydroxy-3-(4-(5-(3,4,5-trichlorophenyl)-5-(trifluoromethyl)-4,5-dihydroisoxazol-3-yl)phenyl)azetidine-1-carboxylate;
(S)-tert-butyl 3-(4-(5-(3,4-dichloro-5-(trifluoromethyl)phenyl)-5-(trifluoromethyl)-4,5-dihydroisoxazol-3-yl)phenyl)-3-hydroxyazetidine-1-carboxylate;
(R)-tert-butyl 3-(4-(5-(3,5-dichloro-4-fluorophenyl)-5-(trifluoromethyl)-4,5-dihydroisoxazol-3-yl)phenyl)-3-hydroxyazetidine-1-carboxylate;
(R)-tert-butyl 3-(4-(5-(3,5-dichlorophenyl)-5-(trifluoromethyl)-4,5-dihydroisoxazol-3-yl)phenyl)-3-hydroxyazetidine-1-carboxylate;
(R)-tert-butyl 3-hydroxy-3-(4-(5-(3,4,5-trichlorophenyl)-5-(trifluoromethyl)-4,5-dihydroisoxazol-3-yl)phenyl)azetidine-1-carboxylate;
(R)-tert-butyl 3-(4-(5-(3,4-dichloro-5-(trifluoromethyl)phenyl)-5-(trifluoromethyl)-4,5-dihydroisoxazol-3-yl)phenyl)-3-hydroxyazetidine-1-carboxylate;
(S)-tert-butyl 3-(4-(5-(3,5-dichloro-4-fluorophenyl)-5-(trifluoromethyl)-4,5-dihydroisoxazol-3-yl)phenyl)-3-fluoroazetidine-1-carboxylate;
(S)-tert-butyl 3-(4-(5-(3,5-dichlorophenyl)-5-(trifluoromethyl)-4,5-dihydroisoxazol-3-yl)phenyl)-3-fluoroazetidine-1-carboxylate;
(S)-tert-butyl 3-fluoro-3-(4-(5-(3,4,5-trichlorophenyl)-5-(trifluoromethyl)-4,5-dihydroisoxazol-3-yl)phenyl)azetidine-1-carboxylate;
(S)-tert-butyl 3-(4-(5-(3,4-dichloro-5-(trifluoromethyl)phenyl)-5-(trifluoromethyl)-4,5-dihydroisoxazol-3-yl)phenyl)-3-fluoroazetidine-1-carboxylate;
(R)-tert-butyl 3-(4-(5-(3,5-dichloro-4-fluorophenyl)-5-(trifluoromethyl)-4,5-dihydroisoxazol-3-yl)phenyl)-3-fluoroazetidine-1-carboxylate;
(R)-tert-butyl 3-(4-(5-(3,5-dichlorophenyl)-5-(trifluoromethyl)-4,5-dihydroisoxazol-3-yl)phenyl)-3-fluoroazetidine-1-carboxylate;
(R)-tert-butyl 3-fluoro-3-(4-(5-(3,4, 5-trichlorophenyl)-5-(trifluoromethyl)-4,5-dihydroisoxazol-3-yl)phenyl)azetidine-1-carboxylate;
(R)-tert-butyl 3-(4-(5-(3,4-dichloro-5-(trifluoromethyl)phenyl)-5-(trifluoromethyl)-4,5-dihydroisoxazol-3-yl)phenyl)-3-fluoroazetidine-1-carboxylate;
(S)-benzyl 3-(4-(5-(3,5-dichloro-4-fluorophenyl)-5-(trifluoromethyl)-4,5-dihydroisoxazol-3-yl)phenyl)-3-hydroxyazetidine-1-carboxylate;
(S)-benzyl 3-(4-(5-(3,5-dichlorophenyl)-5-(trifluoromethyl)-4,5-dihydroisoxazol-3-yl)phenyl)-3-hydroxyazetidine-1-carboxylate;
(S)-benzyl 3-hydroxy-3-(4-(5-(3,4,5-trichlorophenyl)-5-(trifluoromethyl)-4,5-dihydroisoxazol-3-yl)phenyl)azetidine-1-carboxylate;
(S)-benzyl 3-(4-(5-(3,4-dichloro-5-(trifluoromethyl)phenyl)-5-(trifluoromethyl)-4,5-dihydroisoxazol-3-yl)phenyl)-3-hydroxyazetidine-1-carboxylate;
(R)-benzyl 3-(4-(5-(3,5-dichloro-4-fluorophenyl)-5-(trifluoromethyl)-4,5-dihydroisoxazol-3-yl)phenyl)-3-hydroxyazetidine-1-carboxylate;
(R)-benzyl 3-(4-(5-(3,5-dichlorophenyl)-5-(trifluoromethyl)-4,5-dihydroisoxazol-3-yl)phenyl)-3-hydroxyazetidine-1-carboxylate;
(R)-benzyl 3-hydroxy-3-(4-(5-(3,4,5-trichlorophenyl)-5-(trifluoromethyl)-4,5-dihydroisoxazol-3-yl)phenyl)azetidine-1-carboxylate;
(R)-benzyl 3-(4-(5-(3,4-dichloro-5-(trifluoromethyl)phenyl)-5-(trifluoromethyl)-4,5-dihydroisoxazol-3-yl)phenyl)-3-hydroxyazetidine-1-carboxylate;
(S)-benzyl 3-(4-(5-(3,5-dichloro-4-fluorophenyl)-5-(trifluoromethyl)-4,5-dihydroisoxazol-3-yl)phenyl)-3-fluoroazetidine-1-carboxylate;
(S)-benzyl 3-(4-(5-(3,5-dichlorophenyl)-5-(trifluoromethyl)-4,5-dihydroisoxazol-3-yl)phenyl)-3-fluoroazetidine-1-carboxylate;
(S)-benzyl 3-fluoro-3-(4-(5-(3,4, 5-trichlorophenyl)-5-(trifluoromethyl)-4,5-dihydroisoxazol-3-yl)phenyl)azetidine-1-carboxylate;
(S)-benzyl 3-(4-(5-(3,4-dichloro-5-(trifluoromethyl)phenyl)-5-(trifluoromethyl)-4,5-dihydroisoxazol-3-yl)phenyl)-3-fluoroazetidine-1-carboxylate;
(R)-benzyl 3-(4-(5-(3,5-dichloro-4-fluorophenyl)-5-(trifluoromethyl)-4,5-dihydroisoxazol-3-yl)phenyl)-3-fluoroazetidine-1-carboxylate;
(R)-benzyl 3-(4-(5-(3,5-dichlorophenyl)-5-(trifluoromethyl)-4,5-dihydroisoxazol-3-yl)phenyl)-3-fluoroazetidine-1-carboxylate;
(R)-benzyl 3-fluoro-3-(4-(5-(3,4,5-trichlorophenyl)-5-(trifluoromethyl)-4,5-dihydroisoxazol-3-yl)phenyl)azetidine-1-carboxylate;
(R)-benzyl 3-(4-(5-(3,4-dichloro-5-(trifluoromethyl)phenyl)-5-(trifluoromethyl)-4,5-dihydroisoxazol-3-yl)phenyl)-3-fluoroazetidine-1-carboxylate;
(S)-3-(4-(1-benzhydryl-3-hydroxyazetidin-3-yl)phenyl)-5-(3,5-dichloro-4-fluorophenyl)-5-(trifluoromethyl)-4,5-dihydroisoxazole;
(S)-3-(4-(1-benzhydryl-3-hydroxyazetidin-3-yl)phenyl)-5-(3,5-dichlorophenyl)-5-(trifluoromethyl)-4,5-dihydroisoxazole;
(S)-3-(4-(1-benzhydryl-3-hydroxyazetidin-3-yl)phenyl)-5-(3,4,5-trichlorophenyl)-5-(trifluoromethyl)-4,5-dihydroisoxazole;
(S)-3-(4-(1-benzhydryl-3-hydroxyazetidin-3-yl)phenyl)-5-(3,4-dichloro-5-(trifluoromethyl)phenyl)-5-(trifluoromethyl)-4,5-dihydroisoxazole;
(R)-3-(4-(1-benzhydryl-3-hydroxyazetidin-3-yl)phenyl)-5-(3,5-dichloro-4-fluorophenyl)-5-(trifluoromethyl)-4,5-dihydroisoxazole;
(R)-3-(4-(1-benzhydryl-3-hydroxyazetidin-3-yl)phenyl)-5-(3,5-dichlorophenyl)-5-(trifluoromethyl)-4,5-dihydroisoxazole;
(R)-3-(4-(1-benzhydryl-3-hydroxyazetidin-3-yl)phenyl)-5-(3,4,5-trichlorophenyl)-5-(trifluoromethyl)-4,5-dihydroisoxazole;
(R)-3-(4-(1-benzhydryl-3-hydroxyazetidin-3-yl)phenyl)-5-(3,4-dichloro-5-(trifluoromethyl)phenyl)-5-(trifluoromethyl)-4,5-dihydroisoxazole;
(S)-3-(4-(1-benzhydryl-3-fluoroazetidin-3-yl)phenyl)-5-(3,5-dichloro-4-fluorophenyl)-5-(trifluoromethyl)-4,5-dihydroisoxazole;
(S)-3-(4-(1-benzhydryl-3-fluoroazetidin-3-yl)phenyl)-5-(3,5-dichlorophenyl)-5-(trifluoromethyl)-4,5-dihydroisoxazole;
(S)-3-(4-(1-benzhydryl-3-fluoroazetidin-3-yl)phenyl)-5-(3,4,5-trichlorophenyl)-5-(trifluoromethyl)-4,5-dihydroisoxazole;
(S)-3-(4-(1-benzhydryl-3-fluoroazetidin-3-yl)phenyl)-5-(3,4-dichloro-5-(trifluoromethyl)phenyl)-5-(trifluoromethyl)-4,5-dihydroisoxazole;
(R)-3-(4-(1-benzhydryl-3-fluoroazetidin-3-yl)phenyl)-5-(3,5-dichloro-4-fluorophenyl)-5-(trifluoromethyl)-4,5-dihydroisoxazole;
(R)-3-(4-(1-benzhydryl-3-fluoroazetidin-3-yl)phenyl)-5-(3,5-dichlorophenyl)-5-(trifluoromethyl)-4,5-dihydroisoxazole;
(R)-3-(4-(1-benzhydryl-3-fluoroazetidin-3-yl)phenyl)-5-(3,4,5-trichlorophenyl)-5-(trifluoromethyl)-4,5-dihydroisoxazole;
(R)-3-(4-(1-benzhydryl-3-fluoroazetidin-3-yl)phenyl)-5-(3,4-dichloro-5-(trifluoromethyl)phenyl)-5-(trifluoromethyl)-4,5-dihydroisoxazole;
(S)-3-(4-(5-(3,5-dichloro-4-fluorophenyl)-5-(trifluoromethyl)-4,5-dihydroisoxazol-3-yl)phenyl)azetidin-3-ol;
(S)-3-(4-(5-(3,5-dichlorophenyl)-5-(trifluoromethyl)-4,5-dihydroisoxazol-3-yl)phenyl)azetidin-3-ol;
(S)-3-(4-(5-(3,4,5-trichlorophenyl)-5-(trifluoromethyl)-4,5-dihydroisoxazol-3-yl)phenyl)azetidin-3-ol;
(S)-3-(4-(5-(3,4-dichloro-5-(trifluoromethyl)phenyl)-5-(trifluoromethyl)-4,5-dihydroisoxazol-3-yl)phenyl)azetidin-3-ol;
(R)-3-(4-(5-(3,5-dichloro-4-fluorophenyl)-5-(trifluoromethyl)-4,5-dihydroisoxazol-3-yl)phenyl)azetidin-3-ol;
(R)-3-(4-(5-(3,5-dichlorophenyl)-5-(trifluoromethyl)-4,5-dihydroisoxazol-3-yl)phenyl)azetidin-3-ol;
(R)-3-(4-(5-(3,4,5-trichlorophenyl)-5-(trifluoromethyl)-4,5-dihydroisoxazol-3-yl)phenyl)azetidin-3-ol;
(R)-3-(4-(5-(3,4-dichloro-5-(trifluoromethyl)phenyl)-5-(trifluoromethyl)-4,5-dihydroisoxazol-3-yl)phenyl)azetidin-3-ol;
(S)-5-(3,5-dichloro-4-fluorophenyl)-3-(4-(3-fluoroazetidin-3-yl)phenyl)-5-(trifluoromethyl)-4,5-dihydroisoxazole;
(S)-5-(3,5-dichlorophenyl)-3-(4-(3-fluoroazetidin-3-yl)phenyl)-5-(trifluoromethyl)-4,5-dihydroisoxazole;
(S)-3-(4-(3-fluoroazetidin-3-yl)phenyl)-5-(3,4,5-trichlorophenyl)-5-(trifluoromethyl)-4,5-dihydroisoxazole;
(S)-5-(3,4-dichloro-5-(trifluoromethyl)phenyl)-3-(4-(3-fluoroazetidin-3-yl)phenyl)-5-(trifluoromethyl)-4,5-dihydroisoxazole;
(R)-5-(3,5-dichloro-4-fluorophenyl)-3-(4-(3-fluoroazetidin-3-yl)phenyl)-5-(trifluoromethyl)-4,5-dihydroisoxazole;
(R)-5-(3,5-dichlorophenyl)-3-(4-(3-fluoroazetidin-3-yl)phenyl)-5-(trifluoromethyl)-4,5-dihydroisoxazole;
(R)-3-(4-(3-fluoroazetidin-3-yl)phenyl)-5-(3,4,5-trichlorophenyl)-5-(trifluoromethyl)-4,5-dihydroisoxazole; and
(R)-5-(3,4-dichloro-5-(trifluoromethyl)phenyl)-3-(4-(3-fluoroazetidin-3-yl)phenyl)-5-(trifluoromethyl)-4,5-dihydroisoxazole.

12. A compound selected from the group consisting of 3-(4-acetyl-phenyl)-3-fluoro-azetidine-1-carboxylic acid tert-butyl ester;
(Z)-tert-butyl 3-(4-(3-(3,5-dichloro-phenyl)-4,4,4-trifluoro-but-2-enoyl)phenyl)-3-fluoroazetidine-1-carboxylate;
(Z)-tert-butyl 3-(4-(3-(3, 5-dichloro-4-fluoro-phenyl)-4,4,4-trifluoro-but-2-enoyl)-phenyl)-3-fluoro-azetidine-1-carboxylate;
(Z)-tert-butyl 3-(4-(3-(3,4,5-trichloro-phenyl)-4,4,4-trifluoro-but-2-enoyl)-phenyl)-3-fluoro-azetidine-1-carboxylate; and
(R)-tert-butyl 3-(4-(5-(3,5-dichloro-4-fluorophenyl)-5-(trifluoromethyl)-4,5-dihydroisoxazol-3-yl)phenyl)-3-fluoroazetidine-1-carboxylate.

13. A compound of claim 12 selected from the group consisting of
(Z)-tert-butyl 3-(4-(3-(3, 5-dichloro-phenyl)-4,4,4-trifluoro-but-2-enoyl)phenyl)-3-fluoroazetidine-1-carboxylate;
(Z)-tert-butyl 3-(4-(3-(3, 5-dichloro-4-fluoro-phenyl)-4,4,4-trifluoro-but-2-enoyl)-phenyl)-3-fluoro-azetidine-1-carboxylate; and
(Z)-tert-butyl 3-(4-(3-(3,4, 5-trichloro-phenyl)-4,4,4-trifluoro-but-2-enoyl)-phenyl)-3-fluoro-azetidine-1-carboxylate.

14. A compound of claim 12 which is (R)-tert-butyl 3-(4-(5-(3,5-dichloro-4-fluorophenyl)-5-(trifluoromethyl)-4,5-dihydroisoxazol-3-yl)phenyl)-3-fluoroazetidine-1-carboxylate.
